(19) United States
(12) Patent Application Publication
(10) Pub. No.: US 2020/0033346 A1
(43) Pub. Date: Jan. 30, 2020

(54) DIAGNOSTIC AND THERAPEUTIC METHODS FOR KRAS POSITIVE CANCERS

(71) Applicant: The Board of Trustees of the Leland Stanford Junior University, Stanford, CA (US)

(72) Inventors: Arvin Gouw, San Francisco, CA (US); Dean W. Felsher, San Mateo, CA (US); Alice Fan, Mountain View, CA (US)

(21) Appl. No.: 16/491,417

(22) PCT Filed: Mar. 16, 2018
(86) PCT No.: PCT/US2018/023017

§ 371 Date: Sep. 5, 2019

Related U.S. Application Data
(60) Provisional application No. 62/480,044, filed on Mar. 31, 2017.
(60) Provisional application No. 62/472,447, filed on Mar. 16, 2017.

(51) Int. Cl. G01N 33/574; G01N 33/573; G01N 33/92

(52) U.S. Cl. CPC G01N 33/57407; G01N 33/573; G01N 2333/912; G01N 2333/82; G01N 33/92

(57) ABSTRACT

Methods are provided for the detection and treatment of cancers having a KRAS mutation, which KRAS mutation may drive tumorigenesis in the cancer. In some embodiments the KRAS⁺ cancer is a lung adenocarcinoma.

### CROSS REFERENCE

This application claims benefit of U.S. Provisional Patent Application No. 62/472,447, filed Mar. 16, 2017 and U.S. Provisional Patent Application No. 62/480,044, filed Mar. 31, 2017, which applications are incorporated herein by reference in its entirety.

### BACKGROUND OF THE INVENTION

Treatment for cancer patients has historically consisted of systemic cytotoxic chemotherapy, radiation therapy, and surgery. Now, improved understanding of the molecular pathways that drive malignancies has led to the development of agents that target specific molecular pathways in malignant cells, as well as providing an improved ability to identify patients that will benefit from a specific therapy. Many established targeted therapies are administered as orally available small molecule kinase inhibitors, but targeted therapy can also be administered intravenously in the form of monoclonal antibodies or small molecules.

The identification of oncogenic activation of particular tyrosine kinases in some tumors, for example mutations in the epidermal growth factor receptor (EGFR) or rearrangements of the anaplastic lymphoma kinase (ALK) gene or ROS1 gene, has led to a paradigm shift and the development of specific molecular treatments for patients. Furthermore, the identification of these patient subsets has led to an ongoing effort to identify biomarkers and treatments that can be used for other subsets of patients.

The most useful biomarkers for predicting the efficacy of targeted therapy are somatic genome alterations known as “driver mutations”. These mutations occur in cancer cells within genes encoding for proteins critical to cell growth and survival. Many other recurrent molecular alteration that are much less essential to maintain the oncogenic phenotype are often referred to as “passenger mutations”.

Driver mutations are typically transformative, which means that they initiate the evolution of a noncancerous cell to malignancy. In addition, driver mutations often impart an oncogene-addicted biology to the transformed cell, meaning that the mutated protein engenders reliance within the cancer cell to receive a signal from the driver in order to survive. Oncogene addiction make driver mutations good biomarkers for selecting patients for targeted therapies. The extreme reliance of crucial downstream growth and survival pathways in the cell upon a single upstream signal make the cancer susceptible to down-regulation of signal originating from the driver.

In lung adenocarcinoma, as well as with other malignancies, matching a specific targeted drug to the identified driver mutation for an individual patient has resulted in significantly improved therapeutic efficacy, often in conjunction with decreased toxicity. Screening for driver mutations thus has become an increasingly standard part of the diagnostic work-up, and the resultant information is useful in choosing between standard chemotherapy in the absence of a targetable driver mutation versus targeted therapies.

Methods for screening patients for driver mutations and other abnormalities are continually evolving, and there is no single standard platform for testing. Features that make a platform clinically useful are fast turnaround time (two weeks or less); cost efficiency; ability to be performed on clinically available samples; and semi-automation, eliminating reliance upon a single operator. Techniques used commonly in the clinical setting include genetic sequencing for the presence of mutations; allele-specific testing to analyze DNA for a predefined mutation; next generation sequencing; fluorescence in situ (FISH) to detect gene translocations, amplifications, and other rearrangements; immunohistochemistry; and analysis of circulating tumor DNA.

Activating KRAS mutations are observed in a number of epithelial cancers, including, for example, colorectal cancer, NSCLC, and pancreatic ductal adenocarcinoma (PDAC). As a membrane-bound intracellular GTPase, the RAS family of proteins is a central mediator of the MAPK, signal transducer and activator of transcription (STAT), and phosphoinositide 3-kinase (PI3K) signaling pathways, which together control cell proliferation and apoptosis. Oncogenic RAS mutations, most commonly those corresponding to missense substitutions in codons 12, 13, or 61, cause constitutive activity of RAS independent of upstream signals by impairing the function of the RAS GTPase.

The presence of a KRAS mutation has been associated with response or resistance to particular therapies. KRAS mutations may sensitize tumors to antifolates, while conferring resistance to agents such as cetuximab and other EGFR inhibitors. A current focus of targeted therapeutics for patients with KRAS-mutated lung cancer is against downstream effectors of activated KRAS, including MEK inhibition with trametinib, and MEK inhibition with selumetinib. Efforts to inhibit RAS directly have so far not been successful.

Improved methods for screening small numbers of cancer cells and identification of patient subpopulations is of great clinical interest. The present invention addresses this need.

### SUMMARY OF THE INVENTION

Methods are provided for the detection and treatment of cancers having a KRAS mutation, which KRAS mutation may drive tumorigenesis in the cancer. Such cancers may be referred to herein as “KRAS⁺ cancer”. In some embodiments the KRAS⁺ cancer is a lung adenocarcinoma.

It is shown herein that KRAS⁺ cancer cells can be distinguished from KRAS negative cancer cells, as well as from normal counterpart tissue through one or more of (a) detecting upregulated gene expression of fatty acid synthase (FASN); (b) detecting altered ERK1 phosphoisoforms; and (c) detecting induction of a unique lipid signature. Individuals may be selected for therapy by determining the KRAS+ phenotype of the cancer cells. It is further shown that proliferation of KRAS⁺ lung adenocarcinoma cells is suppressed by inhibition of FASN, thereby providing a targeted therapy.

In one embodiment a nanofluidic proteomic immunoassay (NIA) is applied to quantify ERK1 phosphoisoforms in a small amount of lysate from a tumor suspected of being a KRAS⁺ tumor, including KRAS⁺ lung adenocarcinoma cells. By NIA, ERK1 versus ERK2 protein activation allowed distinguishing between KRAS positive and negative tumors in clinical specimens. Specifically, it is found that the KRAS+ tumors have significantly increased levels of ppERK1 and pERK1 when compared to total ERK protein levels, and relative to a normal tissue sample or KRAS negative cancer. Samples of interest for NIA include blood or solid tumor microbiopsy samples, such as fine needle aspirate (FNA) or circulating tumor cells. Samples may be taken at a single timepoint, or may be taken at multiple timepoints. Samples may be as small as 100,000 cells, as small as 5000 cells, as small as 1000 cells, as small as 100 cells, as small as 50 cells, as small as 25 cells or less. The NIA detection method combines isoelectric protein focusing and antibody detection in a nanofluidic system. In some embodiments of the invention, the NIA detection is performed on a sample that has been frozen, where the cells are lysed after thawing. Blood cells may be retained in the sample to reduce variability. Analysis may be performed for up to 60 minutes following sample obtainment, provided the samples are maintained on ice. Because NIA only need minimal amounts of specimen, the analysis is minimally invasive, allowing for example serial protein profiles to be obtained before and after initiating treatment, allowing the determination of predictive protein biomarkers by quantifying early changes in protein activity in patients starting treatment; and the like.

In one embodiment, mass spectrometry, including without limitation desorption electrospray ionization mass spectrometry imaging (DESI-MSI) is performed to analyze KRAS driven metabolism in a cancer cell suspected or known to be a KRAS⁺ cancer cell, including lung adenocarcinoma cells. The total and relative abundances of a number of lipid species are significantly lower in normal tissue than in the cancer tissues.

Differences in the NIA or DESI mass spectra extracted from cancer cells may be compared to normal cells, KRAS⁻ cancer cells, a reference of known KRAS⁺ cancer cells, and the like. Multiple samples may be obtained and analyzed from an individual over time, including an individual treated with a therapeutic regimen for treatment of the cancer. Multiple samples may also be obtained and analyzed over a patient cohort group, for example in the context of clinical trials.

In other embodiments, methods are provided for treatment of KRAS+ lung adenocarcinoma. The cancer may be analyzed by the methods described herein for determination of a KRAS+ phenotype prior to treatment. The cancer may be analyzed over the course of treatment by the methods described herein to determine the effectiveness of therapy with respect to markers indicative of KRAS-driven tumorigenesis. Methods of treatment provide for administration of an effective dose of an inhibitor of fatty acid synthase activity, or fatty acid synthase expression to a patient in need thereof. As shown here, inhibition of FASN suppresses the proliferation of human KRAS⁺ lung cancer cells. In some embodiments, an inhibitor of FASN is provided in a combination therapy with a second therapeutic regimen, for example one or more of surgery, chemotherapy, radiation therapy, immune-oncology therapy, targeted anti-tumor antibody therapy, and the like. The contacting of a cancer cells may be performed in vivo, e.g. for therapeutic purposes, and in vitro, e.g. for screening assays and the like.

In some aspects, the present disclosure provides a method of determining if a tumor of a patient is driven by a KRAS mutation (KRAS⁺), the method comprising: obtaining a sample of a tumor suspected of being KRAS⁺; and performing one or both of: a nanofluidic proteomic immunoassay (NIA) for ERK phosphoisoforms; and desorption electrospray ionization mass spectrometry imaging (DESI-MSI) for lipid species in the region of from about m/z region 700-1000 and/or about m/z 200-400; determining whether the sample displays altered ERK1 isoforms and/or altered lipid species relative to a KRAS⁻ tumor or normal tissue; wherein a KRAS⁺ tumor displays altered ERK1 isoforms and/or altered lipid species relative to a KRAS⁻ tumor or normal tissue; and providing the determination to the patient.

In some embodiments, the method further comprises treating the patient in accordance with the determination. The tumor may be lung adenocarcinoma. In some embodiments, the sample is a biopsy sample. In some embodiments, the biopsy sample is a tumor cell sample of less than 100,000 cells. In some embodiments, the biopsy sample is a fine needle aspirate sample. In some embodiments, the control tissue is a sample from the same tumor at a different time point. Multiple time points from a single tumor may be compared. In some embodiments, the cellular sample was previously frozen.

In some embodiments, the NIA detects significantly increased levels of ppERK1 and pERK1 when compared to total ERK protein levels for a KRAS⁺ tumor. In some embodiments, the DESI-MSI detects significantly increased levels of complex glycerophospholipids and free fatty acids for a KRAS⁺ tumor. The patient may be treated with an inhibitor of fatty acid synthase (FASN). In some embodiments, the inhibitor is administered in combination with a second therapeutic regimen. For example, the inhibitor of FASN may be cerulenin.

In some embodiments, the method further comprises determining whether a sample from the patient displays altered ERK1 isoforms and/or altered lipid species relative to a KRAS⁻ tumor or normal tissue at two or more time points over the course of treatment to determine the effectiveness of therapy with respect to markers indicative of KRAS-driven tumorigenesis.

In some aspects, the present disclosure provides a method for identifying a subject with a KRAS⁺ cancer, the method comprising: performing a nanofluidic proteomic immunoassay (NIA) and/or a desorption electrospray ionization mass spectrometry imaging (DESI-MSI) on a clinical sample obtained from a subject; and measuring ERK1 phosphoisoforms and/or lipid species in the clinical sample.

In some embodiments, the clinical sample has significantly increased levels of ppERK1 and pERK1 when compared to total ERK protein levels. In some embodiments, the clinical sample has significantly increased levels of ppERK1 and pERK1 when compared to a normal tissue sample or KRAS⁻ cancer. In some embodiments, performing the DESI-MSI involves detecting lipid species in a region ranging from about m/z region 700-1000 and/or about m/z 200-400. The clinical sample may display altered lipid species relative to a normal tissue sample or KRAS⁻ cancer. In some embodiments, the clinical sample has increased relative and/or total abundances of m/z 745.5034, PG(18:1/16:1), m/z 747.5190, as PG(18:1/16:0), m/z 793.5023, PG(18:2/20:4), and m/z 865.5034, PG(22:6/22:6). In some embodiments, the DESI-MSI detects significantly increased levels of complex glycerophospholipids and free fatty acids for a KRAS⁺ tumor.

In some embodiments, the clinical sample is a blood sample. In some embodiments, the clinical sample is a biopsy sample. The biopsy sample may be obtained from a tumor. In some embodiments, the clinical sample comprises less than 100,000 cells. The clinical sample may comprise less than 1,000 cells. The clinical sample may comprise less than 100 cells.

In some embodiments, the clinical sample is obtained by fine needle aspiration. In some embodiments, the clinical sample is a fine needle aspirate (FNA) that is sampled in vivo. In some embodiments, the method further comprises comparing the FNA with an adjacent non-tumor tissue. In some embodiments, the subject is diagnosed with lung adenocarcinoma. In some embodiments, the subject is diagnosed with kidney cancer. The method may further comprise performing a second NIA and/or a second DESI-MSI from the same tumor at a different time point.

In some embodiments, the clinical sample was previously frozen. The clinical sample may have been previously maintained on ice for greater than 30 minutes prior to performing the NIA and/or the DESI-MSI.

In some aspects, the present disclosure provides a method of treating or reducing a KRAS⁺ cancer in a subject in need thereof, the method comprising: performing a nanofluidic proteomic immunoassay (NIA) and/or a desorption electrospray ionization mass spectrometry imaging (DESI-MSI) on a clinical sample obtained from a location on the subject; measuring ERK1 phosphoisoforms and/or lipid species in the clinical sample at a first time point; performing a second NIA and/or a second DESI-MSI on the clinical sample obtained from approximately the same location on the subject after the subject has been treated with an effective amount of an anti-cancer agent; and measuring ERK1 phosphoisoforms and/or lipid species in the clinical sample obtained from approximately the same location on the subject after the subject has been treated with an anti-cancer agent at a second time point.

In some embodiments, the anti-cancer agent is a fatty acid synthase inhibitor. In some embodiments, the anti-cancer agent is lipogenesis enzyme inhibitor. In some embodiments, the method further comprises placing the patient on a treatment regimen, wherein the treatment regimen comprises administering an effective amount of an anti-cancer therapeutic for at least 1 month. In some embodiments, the method further comprises maintaining, adjusting, or stopping the treatment regimen based on the ERK1 phosphoisoforms and/or the lipid species in the clinical sample obtained from approximately the same location on the subject after the subject has been treated with the anti-cancer agent, wherein a change in the ERK1 phosphoisoforms and/or the lipid species indicates a response to the treatment regimen. The clinical sample may have significantly increased levels of ppERK1 and pERK1 when compared to total ERK protein levels at the first time point. The clinical sample may have significantly increased levels of ppERK1 and pERK1 when compared to a normal tissue sample or KRAS⁻ cancer at the first time point. In some embodiments, the levels of ppERK1 and pERK1 are greater at the first time point than the second time point. In some embodiments, performing the DESI-MSI involves detecting lipid species in a region ranging from about m/z region 700-1000 and/or about m/z 200-400. The clinical sample may display altered lipid species relative to a normal tissue sample or KRAS⁻ cancer at the first time point. The clinical sample may have increased relative and/or total abundances of m/z 745.5034, PG(18:1/16:1), m/z 747.5190, as PG(18:1/16:0), m/z 793.5023, PG(18:2/20:4), and m/z 865.5034, PG(22:6/22:6) at the first time point. In some embodiments, the DESI-MSI detects significantly increased levels of complex glycerophospholipids and free fatty acids for a KRAS⁺ tumor at the first time point. The levels of complex glycerophospholipids and free fatty acids may be greater at the first time point than the second time point.

In some embodiments, the clinical sample is a blood sample. In some embodiments, the clinical sample is a biopsy sample. The biopsy sample may be obtained from a tumor. In some embodiments, the clinical sample comprises less than 100,000 cells. The clinical sample may comprise less than 1,000 cells. The clinical sample may comprise less than 100 cells. In some embodiments, the clinical sample is obtained by fine needle aspiration. The clinical sample may be a fine needle aspirate (FNA) that is sampled in vivo. In some embodiments, the method further comprises comparing the FNA with an adjacent non-tumor tissue.

In some embodiments, the subject is diagnosed with lung adenocarcinoma. In some embodiments, the subject is diagnosed with kidney cancer. In some embodiments, the clinical sample was previously frozen. The clinical sample may be previously on ice for greater than 30 minutes prior to performing the NIA and/or the DESI-MSI.

In some embodiments, the subject is human. In some embodiments, the subject is an animal. For example, the animal may be a mouse. In some embodiments, the method further comprises transplanting cancer cells into the animal.

In some aspects, the present disclosure provides a method for treating a disease or disorder in a subject, the method comprising administering to the subject an effective amount of an anti-cancer agent, wherein treatment with the anti-cancer agent is based upon the levels of ppERK1 and pERK1, and/or levels of complex glycerophospholipids and free fatty acids in a clinical sample obtained from the subject, and wherein the levels of ppERK1 and pERK1, and/or levels of complex glycerophospholipids and free fatty acids are elevated in comparison to reference levels. In some embodiments, the levels of ppERK1 and pERK1 are measured by nanofluidic proteomic immunoassay (NIA). In some embodiments, the levels of complex glycerophospholipids and free fatty acids is measured by desorption electrospray ionization mass spectrometry imaging (DESI-MSI), wherein the DESI-MSI involves detecting lipid species in a region ranging from about m/z region 700-1000 and/or about m/z 200-400.

In some embodiments, the disease or disorder is a KRAS⁺ cancer. The KRAS⁺ cancer may be lung carcinoma. The KRAS⁺ cancer may be kidney cancer. In some embodiments, the anti-cancer agent is a fatty acid synthase inhibitor. The anti-cancer agent may be a lipogenesis enzyme inhibitor. In some embodiments, the reference levels are the levels of ppERK1 and pERK1, and/or levels of complex glycerophospholipids and free fatty acids in a KRAS⁻ tumor or normal tissue.

In some embodiments, the clinical sample is a blood sample. In some embodiments, the clinical sample is a biopsy sample. The biopsy sample may be obtained from a tumor. In some embodiments, the clinical sample comprises less than 100,000 cells. In some embodiments, the clinical sample comprises less than 1,000 cells. The clinical sample may comprise less than 100 cells. The clinical sample may be obtained by fine needle aspiration. In some embodiments, the clinical sample was previously frozen. In some embodiments, the clinical sample was previously on ice for greater than 30 minutes prior to performing the NIA and/or the DESI-MSI.

In some aspects, the present disclosure provides a method of treating or reducing cancer a KRAS⁺ cancer in a subject in need thereof, the method comprising: transplanting cancer cells into a location of an animal; removing a portion of the cancer cells from the location; treating ex vivo the portion with an effective amount of an anti-cancer agent to generate a treated portion; performing a nanofluidic proteomic immunoassay (NIA) and/or a desorption electrospray ionization mass spectrometry imaging (DESI-MSI) on the treated portion; and measuring ERK1 phosphoisoforms and/or lipid species in the portion.

In some embodiments, the animal is a mouse. In some embodiments, the method further comprises performing a nanofluidic proteomic immunoassay (NIA) and/or a desorption electrospray ionization mass spectrometry imaging (DESI-MSI) on the portion; and measuring ERK1 phosphoisoforms and/or lipid species in the portion prior to treating with the anti-cancer agent.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1C Lipogenesis in KRAS-induced mouse lung cancer. FIG. 1A Microarray analysis showing upregulation of fatty acid synthesis genes in lung cancer. FIG. 1B The 15 most statistically significant differentially expressed between normal and KRAS-activated mouse lung tissue. FIG. 1C Gene expression in normal (n=2) and KRAS-activated mouse lung tissue (n=5). Statistical significance by t-test indicated ** for p-value<0.01.

FIG. 2A-2B Relative mRNA expression: FIG. 2A normal vs. human KRAS-associated lung cancer (n=12), and FIG. 2B normal vs. human non-KRAS lung cancer (n=14). Statistical significance by t-test indicated by * for p-value<0.05; ** for p-value<0.01; *** for p-value<0.001.

FIG. 3A-3D NIA ERK protein signatures: FIG. 3A normal vs. human KRAS-associated lung cancer tissue (n=6); FIG. 3B normal vs. human non-KRAS lung cancer tissue (n=6); and percentage of total ERK FIG. 3C in KRAS tumors and FIG. 3D in non-KRAS tumors. Statistical significance by t-test indicated by * for p-value<0.05; ** for p-value<0.01; *** for p-value<0.001.

FIG. 4 DESI mass spectra of lipids: images of several lipid species overexpressed in mouse tumor foci (top panel) compared to normal mouse lung tissue (panel below) and corresponding representative mass spectra.

FIG. 5A-5B Relative mRNA expression of FASN and SCD upon FIG. 5A ERK inhibition by SCH772984 (n=3), and FIG. 5B KRAS inhibition by FTS in human lung cancer cell line (n=3). Error bars represent 95% confidence interval from Student's t-distribution. Statistical significance by unpaired two sample t-test indicated by * for p-value<0.05; ** for p-value<0.01.

FIG. 6A-6B Suppression of proliferation upon inhibition of FASN by cerulenin in human lung cancer cell lines FIG. 6A A549 and FIG. 6B H1299 (n=3 for each cell line). Statistical significance by t-test on day 4 when compared to control indicated by * for p-value<0.05; ** for p-value<0.01; *** for p-value<0.001.

FIG. 7 List of all metabolism genes used in microarray analysis. Supplement to FIG. 1A.

FIG. 8 DESI-MSI instrument setup.

FIG. 9 Tetracycline-based conditional oncogene activation. In the absence of doxycycline (dox), reversible tetracycline transactivating factor (rtTA) is unable to bind tetO sequences therefore oncogene expression does not occur. When dox is added, rtTA binds dox and undergoes a conformational change which permits it to bind tetO sequences and activate oncogene expression. This system is used to conditionally activate oncogene expression in transgenic mouse models.

FIG. 10 DESI-MSI image and representative mass spectra of a solid pattern adenoma focus. H&E staining of the imaged tissue confirms several regions with adenoma, which are marked in red.

FIG. 11 Tandem mass spectrometry data used for identification of molecular ions. Inhibiting the lipogenesis pathway.

FIG. 12 The production of fatty acids can be suppressed by the inhibitor, cerulenin, which inhibits the enzyme, fatty acid synthase (FASN).

FIG. 13 Human lung adenocarcinoma samples.

FIG. 14 Primers used for real-time PCR.

FIG. 15 Intra- and inter-patient variability of ERK isoforms. 39 patients each had 2-3 regions of their kidney tumor sampled by fine needle aspirate. ERK isoforms in each FNA were measured using NIA. Each circle is a tumor FNA (N=91 FNAs), averaged across technical replicates. Samples from each patient are connected by a vertical line. Patients are ordered by the average across samples of ERK2. Across isoforms, the variation across technical replicates has an average standard deviation of 1%. In samples from different regions of the same tumor, the variation between samples has an average standard deviation of 6%. In contrast, the standard deviation of the measured proportions across different patients ranges from 5% to 22% across isoforms.

FIG. 16 is a graphical representation that shows that ERK2 is highly phosphorylated in lung cancer CTCs.

FIG. 17 shows that farnesyl thiosalicylic acid (FTS) blocks Ras binding at plasma membrane.

FIG. 18 shows FTS treatment inhibits ERK activation in vivo (NIA Analysis).

FIG. 19 shows BCL2+ Ras inactivation induces apoptosis in BCL2 lymphoma in vivo (serial FNA's).

FIG. 20 shows the inactivation of BCL2 and ras inhibits tumor growth more effectively than inactivating either oncogene alone.

FIG. 21 shows the inactivation of BCL2 by DOX and RAS by FTS inhibits tumor growth more effectively than inactivating either oncogene alone.

FIG. 22 shows NIA analysis of ERK isoforms in FNAs from transgenic lymphoma.

FIG. 23 shows NIA analysis of ERK isoforms in FNAs from transgenic lymphoma.

FIG. 24 shows ERK data (pre and post atorvastatin treatment).

FIG. 25 shows MEK data (pre and post atorvastatin treatment).

FIG. 26 shows atorvastatin causes significant changes in tri-phospho-MEK1 in four of nine NHL patients.

FIG. 27 shows atorvastatin causes significant changes in di-phospho-MEK1 in four of nine NHL patients.

FIG. 28 shows atorvastatin causes significant changes in Mono-phospho-MEK1 in one of nine NHL patients.

FIG. 29 shows Erk activity in therapeutic response to Rigosertib treatment.

FIG. 30 shows Rigosertib's mechanism of action.

FIG. 31 shows the process for Rigosertib treatment.

FIG. 32 shows that Rigosertib decreases Erk pathway in head & neck squamous cell carcinoma.

### DETAILED DESCRIPTION OF THE EMBODIMENTS

Methods are provided for the detection and treatment of cancers having a KRAS mutation, which KRAS mutation may drive tumorigenesis in the cancer. KRAS⁺ cancer cells can be distinguished from KRAS⁻ cancer cells, as well as from normal counterpart tissue through one or more of (a) detecting upregulated gene expression of fatty acid synthase (FASN); (b) detecting altered ERK1 phosphoisoforms; and (c) detecting induction of a unique lipid signature. Individuals may be selected for therapy by determining the KRAS+ phenotype of the cancer cells. Such treatment may include inhibition of FASN expression or enzyme activity.

### Definitions

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a compound” includes a plurality of such compounds and reference to “the agent” includes reference to one or more agents and equivalents thereof known to those skilled in the art, and so forth. All technical and scientific terms used herein have the same meaning as commonly understood to one of ordinary skill in the art to which this invention belongs unless clearly indicated otherwise.

KRAS. Mutations in KRAS at codons 12 and 13 occur in about 15-50% of NSCLC patients. Approximately 30% to 50% of colorectal tumors are known to have a mutated KRAS gene. The methods provided herein are generally drawn to the phenotypic effects of a KRAS driver in cancer. However, alternative methods include, for example, analysis of the genotype of these genes. Traditional methods for detecting mutations involved screening by direct DNA sequencing of the tumor tissue. Sanger sequencing technology is available in most molecular diagnostic laboratories, and it has the singular advantage of detecting alterations across a gene, including novel variants. Recent methodologies have focused on targeted screening of mutations to achieve more rapid, robust, and sensitive tests. Molecular diagnostic laboratories currently use a variety of methods, including amplification refractory mutation system, pyrosequencing, smart amplification process, high-resolution melting analysis, and restriction fragment length polymorphism, to name a few. These methods all distinguish between mutant and wild-type DNA within the region of interest.

A commercially available test for this purpose is therascreen KRAS RGQ (Rotor-Gene Q) PCR (polymerase chain reaction) Kit. Tests for mutations in codons 12 or 13 of the KRAS gene can be performed on formalin-fixed, paraffin-embedded tissue from the primary tumor or a metastasis.

Lung Adenocarcinoma. Lung adenocarcinoma is a subset of non-small cell lung carcinoma and represent about 35-40% of all lung cancers. Symptoms can include cough, chest discomfort or pain, weight loss, and, less commonly, hemoptysis; however, many patients present with metastatic disease without any clinical symptoms. The diagnosis is typically made by chest x-ray or CT and confirmed by biopsy.

Respiratory epithelial cells require prolonged exposure to cancer-promoting agents and accumulation of multiple genetic mutations before becoming neoplastic (an effect called field carcinogenesis). In some patients with lung cancer, secondary or additional mutations in genes that stimulate cell growth (K-ras, MYC), cause abnormalities in growth factor receptor signaling (EGFR, HER2/neu), and inhibit apoptosis contribute to proliferation of abnormal cells. In addition, mutations that inhibit tumor-suppressor genes (p53, APC) can lead to cancer. Other mutations that may be responsible include the EML-4-ALK translocation and mutations in ROS-1, BRAF, and PI3KCA.

Although oncogenic driver mutations can cause or contribute to lung cancer among smokers, these mutations are particularly likely to be a cause of lung cancer among nonsmokers, and have been primarily identified in adenocarcinoma. In 2014, the Lung Cancer Mutation Consortium (LCMC) found driver mutations in 64% of 733 lung cancers among smokers and nonsmokers (25% K-ras mutations, 17% EGFR mutations, 8% EML-4-ALK, and 2% BRAF mutations).

The clinical behavior of NSCLC is more variable and depends on histologic type, but about 40% of patients will have metastatic disease outside of the chest at the time of diagnosis. Oncogenic driver mutations have been identified primarily in adenocarcinoma, although attempts are being made to identify similar mutations in squamous cell carcinoma.

Conventional treatment for Stage I and II is surgery with or without adjuvant chemotherapy, at Stage IIIA surgery with or without adjuvant chemotherapy or concurrent chemotherapy or radiation therapy, chemotherapy plus radiation therapy and surgery, chemotherapy with surgery, or chemotherapy plus radiation therapy; at Stage IIIB: Radiation therapy with or without chemotherapy; and at Stage IV: Systemic targeted therapy or chemotherapy with or without palliative radiation therapy.

Adjuvant chemotherapy after surgery is now standard practice for patients with stage II or stage III disease and possibly also for patients with stage IB disease and tumors >4 cm. A commonly used chemotherapy regimen is a cisplatin-based doublet (combination of a cisplatin and another chemotherapy drug, such as vinorelbine, docetaxel, paclitaxel). Neoadjuvant (preoperative) chemotherapy in early-stage NSCLC is also commonly used and consists of 4 cycles of a cisplatin-doublet. In patients who cannot receive cisplatin, carboplatin can be substituted.

The 5-yr survival rate varies by stage, from 60 to 70% for patients with stage I disease to <1% for patients with stage IV disease. On average, untreated patients with metastatic NSCLC survive 6 mo, whereas the median survival for treated patients is about 9 mo. Recently, patient survival has improved in both early and later stage NSCLC. Evidence shows improved survival in early-stage disease (stages IB to IIIB) when platinum-based chemotherapy regimens are used after surgical resection. In addition, targeted therapies have improved survival in patients with stage IV disease, in particular patients with an EGFR mutation, EML-4-ALK and ROS-1 translocations.

For tumors bearing an oncogenic driver mutation, inhibitors are used first. In stage IV patients with sensitive EGFR mutations (ie, deletion exon 19, exon 21 L858 mutation), EGFR tyrosine kinase inhibitors (TKIs) may be given as first-line therapy; response rates and progression-free survival are better than those obtained using standard chemotherapy. EGFR TKIs include gefitinib and erlotinib. Patients who have EML-4-ALK translocations should receive crizotinib, an ALK and ROS-1 inhibitor. Patients with ALK mutations can be given alectinib or ceritinib. Patients with ROS-1 mutations can be given crizotinib or erlotinib. Patients with BRAF mutations may benefit from the BRAF inhibitors (eg, vemurafenib). Similarly, patients with PI3K mutations may be expected to respond to PI3K inhibitors, which are being developed. Any of the methods described herein may be used to treat or reduce lung carcinoma.

Kidney Cancer. Kidney cancer is a type of cancer that starts in the cells in the kidney. The two most common types of kidney cancer are renal cell carcinoma (RCC) and transitional cell carcinoma (TCC) (also known as urothelial cell carcinoma) of the renal pelvis. The different types of kidney cancer (such as RCC and TCC) develop in different ways, meaning that the diseases have different long term outcomes, and need to be staged and treated in different ways. RCC is responsible for approximately 80% of primary renal cancers, and TCC accounts the majority of the remainder.

The most common signs and symptoms of kidney cancer are a mass in the abdomen and/or blood in the urine (or hematuria). Other symptoms may include tiredness, loss of appetite, weight loss, a high temperature and heavy sweating, and persistent pain in the abdomen. However, many of these symptoms can be caused by other conditions, and there may also be no signs or symptoms in a person with kidney cancer, especially in the early stages of the disease.

Treatment for kidney cancer depends on the type and stage of the disease. Surgery is the most common treatment as kidney cancer does not often respond to chemotherapy and radiotherapy. Other treatment options include biological therapies such as everolimus, torisel, nexavar, sutent, and axitinib, the use of immunotherapy including interferon and interleukin-2. Any of the methods described herein may be used to treat or reduce kidney cancer.

FASN inhibitors. Cerulenin and C75, both early small-molecule FASN inhibitors, have demonstrated significant antitumor activity. Cerulenin was isolated from Cephalosporium caerulens; it contains an epoxy group that reacts with the ketoacyl synthase domain of FASN. It was one of the first compounds to be found to inhibit FASN in breast cancer cell lines, inducing programmed cell death, and to delay disease progression in a xenograft model of ovarian cancer; its cytotoxic effects are dependent on the level of FASN activity. C75 was designed after cerulenin to overcome its chemical instability. C75 is a weak, irreversible inhibitor of FASN that interacts with the β-ketoacyl synthase, the enoyl reductase and the thioesterase domains. Recently, more potent analogs of C75 have been designed as FASN inhibitors.

Several natural plant-derived polyphenols have been shown to inhibit FASN, including epigallocatechin-3-gallate (EGCG) and the flavonoids luteolin, taxifolin, kaempferol, quercetin and apigenin. One of the best characterized polyphenol FASN inhibitors is EGCG, a natural component of green tea. EGCG is a high micromolar time-dependent inhibitor of FASN ketoacyl reductase domain. Although EGCG is a promiscuous inhibitor targeting multiple signaling pathways, its apoptosis-inducing effect seems to correlate with its activity at FASN. Another compound, luteolin, has the greatest effect on lipogenesis of the polyphenols and inhibits FASN directly. It has structural homology to PI3K inhibitors and has strong antioxidant activity. Recently, more potent analogs of EGCG have been developed.

Orlistat is a US FDA-approved pancreatic lipase inhibitor, originally developed as an anti-obesity drug, and is a potent inhibitor of FASN. Orlistat is an irreversible inhibitor forming a covalent adduct with the active serine of FASN thioesterase domain.

C93 (or FAS93), a synthetic FASN inhibitor designed after the bacterial FabB inhibitor thiolactomycin, was recently developed as part of an effort to overcome C75's lack of potency and side effects. C247 belongs to the same class of compounds as C93 and has also demonstrated efficacy in a transgenic model of breast cancer with no weight-loss side effects. A new orally available FASN inhibitor, FAS31 has also been developed.

High potency FASN inhibitors have been identified through high-throughput screening or medicinal chemistry programs. For example, a research group at Merck developed a series of 3-aryl-4-hydroxyquinolin-2(1H)-one derivatives while another research group at AstraZeneca developed a series of bisamide derivatives as FASN inhibitors. The dibenzenesulfonamide urea GSK837149A was identified as a low, nanomolar FASN inhibitor by high-throughput screening at GlaxoSmithKline. A systematic screening of natural product extracts led to the isolation of platensimycin as a potent inhibitor of bacterial FabF/B with a broad-spectrum Gram-positive antibacterial activity.

NIA. In some embodiments methods are provided for nanofluidic proteomic immunoassay (NIA), including the serial analysis of cancer. NIA detection accurately measure oncoprotein expression and activation in limited clinical specimens, including particularly ERK isoforms that differ in phosphorylation. The NIA detection method combines isoelectric protein focusing and antibody detection in a nanofluidic system. In some embodiments, detection of the presence of ppERK1 and pERK1 isoform is indicative of a KRAS⁺ cancer. The isoform may be detected by NIA, or by conventional methods if sample is not limiting.

Samples may be taken at a single timepoint, or may be taken at multiple timepoints. Samples may be as small as 100,000 cells, as small as 5000 cells, as small as 1000 cells, as small as 500 cells, as small as 100 cells, as small as 50 cells or less. In some embodiments the sample is a fine needle aspirate, (FNA). FNAs are performed at physicians' discretion. This procedure entails inserting a small-gauge needle, usually a 21- to 25-gauge needle, into a mass to remove a cellular sample for microscopic evaluation. The procedure should be performed by using sewing machine-like excursions, while applying minimal negative pressure (No more than 0.5 cc of suction is needed.)

Biopsy samples. Particularly fine needle aspirates (FNA) can be maintained on ice for more than 30 minutes, or more than 60 minutes, usually not more than about 120 minutes following obtention from a patient. For any of the methods described herein, the clinical samples, such as tumor cell samples, can be maintained on ice for more than 30 minutes, or more than 60 minutes, usually not more than about 120 minutes prior to performing the NIA and/or the DESI-MSI to determine the levels and/or ratios of ERK1 phosphoisoforms and/or lipid species. The sample is usually maintained without lysis of cells or red blood cells. The sample, or a lysate thereof, is stable when stored frozen at about −80 degrees C. for long periods of time. Thus in some embodiments of the invention the analysis is performed on a previously frozen sample.

The cells, which may be cells after exposure to an agent or condition of interest, are lysed prior to analysis. Methods of lysis are known in the art, including sonication, non-ionic surfactants, etc. Non-ionic surfactants include the Triton™ family of detergents, e.g. Triton™ X-15; Triton™ X-35; Triton™ X-45; Triton™ X-100; Triton™ X-102; Triton™ X-114; Triton™ X-165, etc. Brij™ detergents are also similar in structure to Triton™ X detergents in that they have varying lengths of polyoxyethylene chains attached to a hydrophobic chain. The Tween™ detergents are nondenaturing, nonionic detergents, which are polyoxyethylene sorbitan esters of fatty acids. Tween™ 80 is derived from oleic acid with a Cis chain while Tween™ 20 is derived from lauric acid with a C₁₂ chain. The zwitterionic detergent, CHAPS, is a sulfobetaine derivative of cholic acid. BICINE (diethylolglycine) is zwitterionic amino acid buffer that may be formulated with CHAPS. This zwitterionic detergent and buffer is useful for membrane protein solubilization when protein activity is important. The surfactant is contacted with the cells for a period of time sufficient to lyse the cells and remove additional adherent cells from the system.

Methods of cellular fractionation are also known in the art. Subcellular fractionation consists of two major steps, disruption of the cellular organization (lysis) and fractionation of the homogenate to separate the different populations of organelles. Such a homogenate can then be resolved by differential centrifugation into several fractions containing mainly (1) nuclei, heavy mitochondria, cytoskeletal networks, and plasma membrane; (2) light mitochondria, lysosomes, and peroxisomes; (3) Golgi apparatus, endosomes and microsomes, and endoplasmic reticulum (ER); and (4) cytosol. Each population of organelles is characterized by size, density, charge, and other properties on which the separation relies.

The isoelectricly focused protein is bound to a specific binding member. For relative ratio measurements, a single, pan-specific antibody that recognizes all isoforms of the protein may be used, for example pan-specific ERK antibody, etc. The total amount of the protein, e.g. ERK2, Erk1, etc. is determined, and NIA is used to calculate the percent that is phosphorylated. NIA generates peaks, and the area of each peak was calculated by dropping verticals to the baseline at the peak start and end, and summing the area between the start and endpoints. For normalized value measurements a similar process is used, but in addition the assay utilizes an antibody for the protein of interest, e.g. pan-specific ERK antibody, and a loading control antibody, e.g. HSP-70 antibody, and the like, for normalization. NIA is utilized to discriminate the different isoforms.

Comparisons may be performed between tissue suspected of being a tumor tissue and a paired normal or on-tumor control tissue, e.g. a suspected lung adenocarcinoma sample, v. an adjacent non-tumor skin sample, and the like. Comparisons may also be performed with reference tumor tissue, with a time series of samples, e.g. before and after treatment, and the like. A ratio may be non-tumor/tumor, or tumor/non-tumor. In some embodiments a ratio provides a more predictive or diagnostic biomarker than a single measurement of tumor or normal.

In some embodiments NIA is used to monitor changes in phosphorylation. The vast majority of phosphorylations occur as a mechanism to regulate the biological activity of a protein and as such are transient. In animal cells serine, threonine and tyrosine are the amino acids subject to phosphorylation. The largest group of kinases are those that phosphorylate either serines or threonines and as such are termed serine/threonine kinases. The ratio of phosphorylation of the three different amino acids is approximately 1000/100/1 for serine/threonine/tyrosine. Although the level of tyrosine phosphorylation is minor, the importance of phosphorylation of this amino acid is profound. As an example, the activity of numerous growth factor receptors is controlled by tyrosine phosphorylation.

DESI-MSI. In one embodiment, desorption electrospray ionization mass spectrometry imaging (DESI-MSI) is performed to analyze KRAS driven metabolism in a cancer cell suspected or known to be a KRAS+ cancer cell, including lung adenocarcinoma cells. DESI-MSI is an established technique for real-time, in situ analysis of tissue metabolism. Tissue sections are bombarded with charged microdroplets containing a 1:1 mixture of dimethylformamide and acetonitrile, which are generated by electrospray, causing lipids and metabolites in the tissue samples to be dissolved and extracted. The continuous impact of the spray on the sample then creates a splash of secondary microdroplets, containing dissolved analytes, which are captured by a mass spectrometer. A two-dimensional chemical map of the tissue section is created based on mass spectrometry analysis.

In the m/z region 700-1000 where most complex glycerophospholipids are observed, an increase in the relative and total abundances of m/z 745.5034, PG(18:1/16:1), m/z 747.5190, as PG(18:1/16:0), m/z 793.5023, PG(18:2/20:4), and m/z 865.5034, PG(22:6/22:6), are detected in KRAS⁺ cancer cells. Changes in the relative and total abundances of free fatty acids, in the m/z 200-400, are also observed, including m/z 255.2339, palmitic acid FA(16:0), m/z 281.2490, oleic acid FA(18:1), m/z 303.2333, arachidonic acid FA(20:4), and m/z 327.2334, docosahexaenoic acid, FA(22:6). The total and relative abundances of these species are significantly lower in normal tissue than in the cancer tissues.

Mammalian species that provide tissue for analysis include canines; felines; equines; bovines; ovines; etc. and primates, particularly humans. Animal models, particularly small mammals, e.g. murine, lagomorpha, etc. may be used for experimental investigations. Animal models of interest include those for models of tumors, immune responsiveness, and the like.

In one embodiment of the invention, the NIA is used to guide selection of patient appropriate agents for therapy by determining whether a cancer is a KRAS⁺ cancer. A particular advantage of the invention is the ability to provide individualized diagnosis, taking advantage of small sample size to assess cancer patterns of expression over time.

The information obtained from NIA or DESI-MSI is used to monitor treatment, modify therapeutic regimens, and to further optimize the selection of therapeutic agents. With this approach, therapeutic and/or diagnostic regimens can be individualized and tailored according to the data obtained at different times over the course of treatment.

A “patient” for the purposes of the present invention includes both humans and other animals, particularly mammals, including pet and laboratory animals, e.g. mice, rats, rabbits, etc. Thus the methods are applicable to both human therapy and veterinary applications. In one embodiment the patient is a mammal, preferably a primate. In other embodiments the patient is human.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer. Subjects may be human, but also include other mammals, particularly those mammals useful as laboratory models for human disease, e.g. mouse, rat, etc.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell. Many types of cancers are known to those of skill in the art, including solid tumors such as carcinomas, sarcomas, glioblastomas, melanomas, lymphomas, myelomas, etc., and circulating cancers such as leukemias. Examples of cancer include but are not limited to, ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supematants, cell lysates, tissue samples, organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprising a cancer cell from a patient can also include non-cancerous cells.

The term “diagnosis” is used herein to refer to the identification of a molecular or pathological state, disease or condition, such as the identification of a molecular subtype of breast cancer, prostate cancer, or other type of cancer.

The term “prognosis” is used herein to refer to the prediction of the likelihood of cancer-attributable death or progression, including recurrence, metastatic spread, and drug resistance, of a neoplastic disease, such as ovarian cancer. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

Treating may refer to any indicia of success in the treatment or amelioration or prevention of an cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

“Concomitant administration” of a cancer therapeutic drug, immune-oncology agent, tumor-directed antibody, etc. in combination with a FASN inhibitor means administration with the FASN inhibitor at such time that both the drug, antibody and the composition of the present invention will have a therapeutic effect. Such concomitant administration may involve concurrent (i.e. at the same time), prior, or subsequent administration of the drug, or antibody with respect to the administration of a compound of the invention. A person of ordinary skill in the art would have no difficulty determining the appropriate timing, sequence and dosages of administration for particular drugs and compositions of the present invention.

As used herein, endpoints for treatment will be given a meaning as known in the art and as used by the Food and Drug Administration.

Overall survival is defined as the time from randomization until death from any cause, and is measured in the intent-to-treat population. Survival is considered the most reliable cancer endpoint, and when studies can be conducted to adequately assess survival, it is usually the preferred endpoint. This endpoint is precise and easy to measure, documented by the date of death. Bias is not a factor in endpoint measurement. Survival improvement should be analyzed as a risk-benefit analysis to assess clinical benefit. Overall survival can be evaluated in randomized controlled studies. Demonstration of a statistically significant improvement in overall survival can be considered to be clinically significant if the toxicity profile is acceptable, and has often supported new drug approval. A benefit of the methods of the invention can include increased overall survival of patients.

Endpoints that are based on tumor assessments include DFS, ORR, TTP, PFS, and time-to-treatment failure (TTF). The collection and analysis of data on these time-dependent endpoints are based on indirect assessments, calculations, and estimates (e.g., tumor measurements). Disease-Free Survival (DFS) is defined as the time from randomization until recurrence of tumor or death from any cause. The most frequent use of this endpoint is in the adjuvant setting after definitive surgery or radiotherapy. DFS also can be an important endpoint when a large percentage of patients achieve complete responses with chemotherapy.

Objective Response Rate. ORR is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study.

Time to Progression and Progression-Free Survival. TTP and PFS have served as primary endpoints for drug approval. TTP is defined as the time from randomization until objective tumor progression; TTP does not include deaths. PFS is defined as the time from randomization until objective tumor progression or death. The precise definition of tumor progression is important and should be carefully detailed in the protocol.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

### Methods

Methods are provided for diagnosis and treating or reducing growth of primary or metastatic cancer, specifically including KRAS-driven epithelial cancers, e.g. lung adenocarcinomas, colorectal carcinomas, etc., particularly lung adenocarcinomas.

KRAS⁺ cancer cells can be distinguished from KRAS negative cancer cells, as well as from normal counterpart tissue through detecting altered ERK1 phosphoisoforms and/or detecting induction of a unique lipid signature. Individuals may be selected for therapy by determining the KRAS+ phenotype of the cancer cells, for example by treatment with an inhibitor of fatty acid synthase, or other inhibitors of lipogenesis enzymes.

In one embodiment a nanofluidic proteomic immunoassay (NIA) is applied to quantify ERK1 phosphoisoforms in a small amount of lysate from a tumor suspected of being a KRAS⁺ tumor, including KRAS⁺ lung adenocarcinoma cells. By NIA, ERK1 versus ERK2 protein activation allowed distinguishing between KRAS positive and negative tumors in clinical specimens. Specifically, it is found that the KRAS+ tumors have significantly increased levels of ppERK1 and pERK1 when compared to total ERK protein levels, and relative to a normal tissue sample or KRAS negative cancer.

In one embodiment, desorption electrospray ionization mass spectrometry imaging (DESI-MSI) is performed to analyze KRAS driven metabolism in a cancer cell suspected or known to be a KRAS+ cancer cell, including lung adenocarcinoma cells. The total and relative abundances of a number of lipid species are significantly lower in normal tissue than in the cancer tissues. Specifically, in the m/z region 700-1000 where most complex glycerophospholipids are observed, an increase in the relative and total abundances of m/z 745.5034, PG(18:1/16:1), m/z 747.5190, as PG(18:1/16:0), m/z 793.5023, PG(18:2/20:4), and m/z 865.5034, PG(22:6/22:6), are detected in KRAS⁺ cancer cells. Changes in the relative and total abundances of free fatty acids, in the m/z 200-400, are also observed, including m/z 255.2339, palmitic acid FA(16:0), m/z 281.2490, oleic acid FA(18:1), m/z 303.2333, arachidonic acid FA(20:4), and m/z 327.2334, docosahexaenoic acid, FA(22:6). The total and relative abundances of these species are significantly lower in normal tissue than in the cancer tissues.

Differences in the NIA or DESI mass spectra extracted from cancer cells may be compared to normal cells, KRAS⁻ cancer cells, a reference of known KRAS⁺ cancer cells, and the like. Multiple samples may be obtained and analyzed from an individual over time, including an individual treated with a therapeutic regimen for treatment of the cancer. Multiple samples may also be obtained and analyzed over a patient cohort group, for example in the context of clinical trials.

In other embodiments, methods are provided for treatment of KRAS+ lung adenocarcinoma. The cancer may be analyzed by the methods described herein for determination of a KRAS+ phenotype prior to treatment. The cancer may be analyzed over the course of treatment by the methods described herein to determine the effectiveness of therapy with respect to markers indicative of KRAS-driven tumorigenesis. Methods of treatment provide for administration of an effective dose of an inhibitor of fatty acid synthase activity, or fatty acid synthase expression to a patient in need thereof. As shown here, inhibition of FASN suppresses the proliferation of human KRAS⁺ lung cancer cells. In some embodiments, an inhibitor of FASN is provided in a combination therapy with one or more of surgery, chemotherapy, radiation therapy, immune-oncology therapy, targeted anti-tumor antibody therapy, and the like. The contacting of a cancer cells may be performed in vivo, e.g. for therapeutic purposes, and in vitro, e.g. for screening assays and the like.

Effective doses of the agent(s) of the present invention for the treatment of cancer, vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

In some embodiments, the therapeutic dosage of each agent may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

In prophylactic applications, a relatively low dosage may be administered at relatively infrequent intervals over a long period of time. Some patients continue to receive treatment for the rest of their lives. In other therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In still other embodiments, methods of the present invention include treating, reducing or preventing tumor growth, tumor metastasis or tumor invasion of cancers including carcinomas, gliomas, etc. For prophylactic applications, pharmaceutical compositions or medicaments are administered to a patient susceptible to, or otherwise at risk of disease in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

Compositions for the treatment of cancer can be administered by parenteral, topical, intravenous, intratumoral, oral, subcutaneous, intraarterial, intracranial, intraperitoneal, intranasal or intramuscular means. A typical route of administration is intravenous or intratumoral, although other routes can be equally effective.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the combined agents described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that compositions of the invention when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions for administration will commonly comprise an antibody or other ablative agent dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

The compositions can be administered for therapeutic treatment. Compositions are administered to a patient in an amount sufficient to substantially ablate targeted cells, as described above. An amount adequate to accomplish this is defined as a “therapeutically effective dose.”, which may provide for an improvement in overall survival rates. Single or multiple administrations of the compositions may be administered depending on the dosage and frequency as required and tolerated by the patient. The particular dose required for a treatment will depend upon the medical condition and history of the mammal, as well as other factors such as age, weight, gender, administration route, efficiency, etc.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

### KRAS Activates Fatty Acid Synthase Resulting in Specific ERK and Lipid Signatures Associated with Lung Adenocarcinoma

KRAS gene mutation causes lung adenocarcinoma. KRAS activation has been associated with altered glucose and glutamine metabolism. Here we show that KRAS activates lipogenesis and this results in distinct proteomic and lipid signatures. By gene expression analysis, KRAS is shown to be associated with a lipogenesis gene signature and specific induction of fatty acid synthase (FASN). Through desorption electrospray ionization mass spectrometry imaging (DESI-MSI), specific changes in lipogenesis and specific lipids are identified. By the nanoimmunoassay (NIA), KRAS is found to activate the protein ERK2, whereas ERK1 activation is found in non-KRAS-associated human lung tumors. The inhibition of FASN by cerulenin, a small-molecule antibiotic, blocked cellular proliferation of KRAS associated lung cancer cells. Hence, KRAS is associated with activation of ERK2, the induction of FASN, and promotion of lipogenesis. FASN may be a novel target for KRAS− associated lung

We studied lung tumors induced by KRAS gene mutation using transgenic mice and human lung specimens. Gene expression analyses show that KRAS induces fatty acid synthase (FASN), promoting lipogenesis. Through desorption electrospray ionization mass spectrometry imaging (DESI-MSI), we found specific lipid modifications in KRAS lung adenocarcinoma. Nanoimmunoassay (NIA) identified specific KRAS-associated phosphoprotein signatures. We showed that KRAS activates the ERK2 protein, whereas non-KRAS lung adenocarcinoma shows elevated ERK1. We inhibited FASN by a small molecule, cerulenin, and this blocked cellular proliferation of KRAS-driven lung cancer cells. FASN inhibitors may thus present promising therapeutic agents for the treatment of KRAS-associated lung adenocarcinoma.

KRAS is a member of the RAS gene subfamily that is commonly mutated in human cancer, including lung adenocarcinoma. The RAS genes encode membrane-localized G proteins that are components of several signaling cascades, including the Raf-MEK-ERK signal transduction pathway. Most RAS mutations in cancer lead to constitutive activation of GTPase, resulting in cellular proliferation.

KRAS activation has been shown to alter glucose and glutamine metabolism. KRAS increases glycolytic flux, decreases oxidative TCA cycle flux, and promotes utilization of glutamine for anabolic pathways. In human pancreatic ductal adenocarcinoma (PDAC) cells, KRAS inhibits glutamate dehydrogenase that converts glutamine-derived glutamate into α-ketoglutarate to fuel the TCA cycle. KRAS also increases expression of GOT1, which converts glutamine-derived aspartate into oxaloacetate in a pathway that generates NADPH. The shift from using glutamine to fuel the TCA cycle to using glutamine in a noncanonical NADPH-generating pathway is essential for the growth of PDAC cells. This results in an increased NADPH/NADP⁺ ratio maintaining redox balance. Changes in metabolism caused by KRAS are thought to play an essential role in the proliferation and survival of cancer. Prior to this study, the regulation of lipogenesis in lung adenocarcinoma by KRAS has not been established.

Mass spectrometry imaging (MSI), a spatially resolved label-free imaging technique, presents an attractive way to visualize the distribution of numerous known and unknown molecular ion species within a tissue of interest without molecule pre-identification. MSI has been extensively investigated as a tool that enables delineation of cancerous tissues from their normal counterparts and categorizes various tumors. Several MSI methods have been employed to delineate and categorize lung neoplasia. Matrix-assisted laser desorption/ionization (MALDI) imaging on tissue microarrays was recently proposed for histopathological subtyping of non-small cell lung cancer into adenocarcinoma and squamous cell carcinoma. Air flow-assisted desorption electrospray ionization (AFADESI) MSI was recently utilized to molecularly visualize post-operative human lung cancer specimens.

In this study, we used DESI-MSI for analyzing KRAS driven metabolism in lung. DESI-MSI is an established, powerful technique for real-time, in situ analysis of tissue metabolism. Tissue sections are bombarded with charged microdroplets containing a 1:1 mixture of dimethylformamide and acetonitrile, which are generated by electrospray, causing lipids and metabolites in the tissue samples to be dissolved and extracted. The continuous impact of the spray on the sample then creates a splash of secondary microdroplets, containing dissolved analytes, which are captured by a mass spectrometer. A two-dimensional chemical map of the tissue section can then be created based on mass spectrometry analysis. DESI-MSI has been extensively used to interrogate the lipid profiles of lymphomas, renal cell carcinoma, thyroid cancer, pancreatic cancer, breast cancer, brain cancer, and other cancerous tissues.

Here, we combined gene expression analysis, nanoscale proteomics and mass spectrometry imaging assays to investigate the relationship between KRAS mutations and lipid signatures in lung cancers. NIA analyses on human patient samples were used to examine KRAS versus non-KRAS lung adenocarcinoma to show that KRAS is associated with phospho-ERK2 induction. Gene expression analyses and DESI-MSI of a murine KRAS lung model show that KRAS induces several genes involved in lipogenesis: sterol regulatory binding protein (SREBP1), fatty acid synthase (FASN), and stearoyl CoA desaturase (SCD). Because the major regulatory site for fatty acid synthesis is on FASN, we decided to inhibit FASN along with others who have shown efficacy in inhibiting FASN in Non-Small Cell Lung Cancer (NSCLC) cells. Notably, our results show that blocking FASN with a therapeutic agent prevents KRAS associated lung cancer cells from proliferating. Thus, blocking FASN provides a novel therapeutic route for treating KRAS-induced lung cancer.

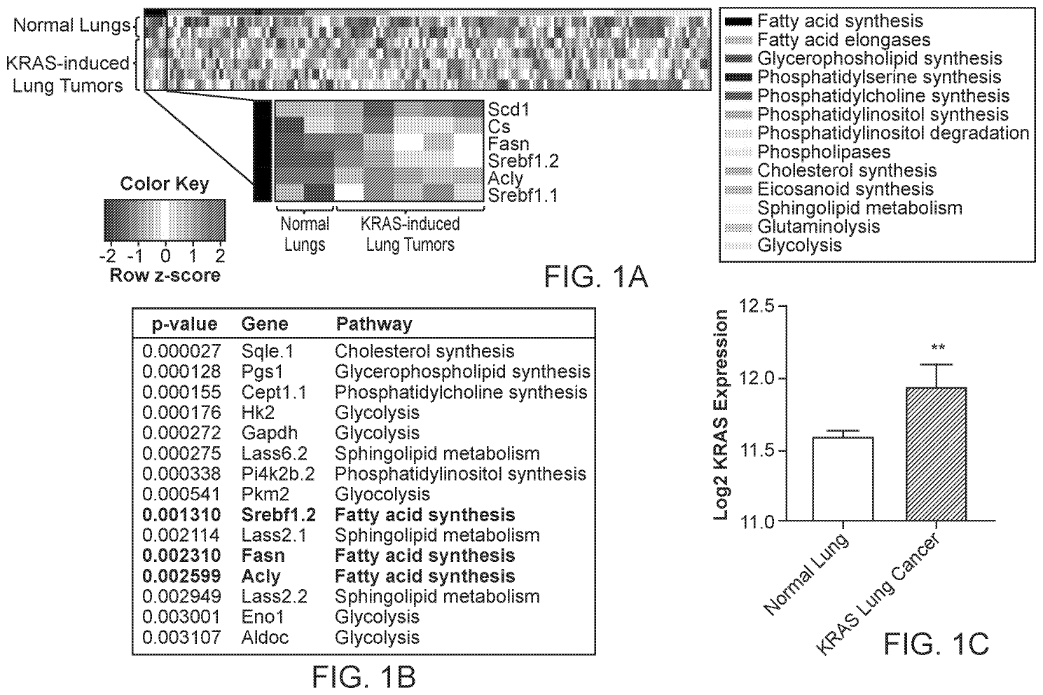

KRAS induces lipogenesis in mouse and human lung adenocarcinoma. The Tet system was used to conditionally express a mutant KRAS gene in mouse lung epithelium resulting in lung adenocarcinoma. We examined 13 metabolic pathways that were identified by 169 microarray probes (FIG. 7) and the results are plotted in the heatmap (FIG. 1A). Gene expression analysis of these tumors showed increased expression of many metabolic genes. We found a majority of the genes in the fatty acid synthesis pathway were in the top 15 most differentially expressed genes (FIG. 1B). Among the lipogenesis genes shown to be induced, we note three particularly important genes. FASN is the regulatory site of fatty acid synthesis, and SCD is the last enzyme in the pathway (FIG. 1C). SREBP is the transcription factor that induces genes involved in fatty acid, mevalonate, and cholesterol syntheses. Induction of FASN, SCD, and SREBP were confirmed by qPCR (FIG. 1C). Thus, KRAS induces lipogenesis pathways in murine lung adenocarcinoma.

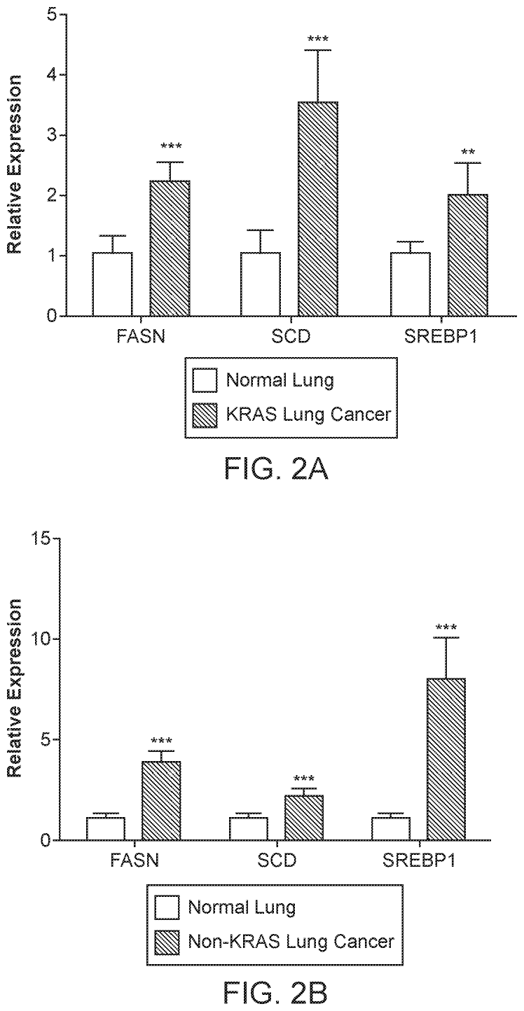

Next, lipogenesis associated genes were examined in human lung adenocarcinoma known to be KRAS positive or negative. Both KRAS positive and negative tumors overexpress lipogenesis genes (FIG. 2A, B). This may reflect the necessity of fatty acid synthesis in highly proliferating cells. SREBP is more elevated in KRAS negative tumors, whereas SCD is more elevated in KRAS positive tumors. FASN is highly upregulated in both. Thus, induction of lipogenesis genes was observed in human KRAS associated lung adenocarcinoma.

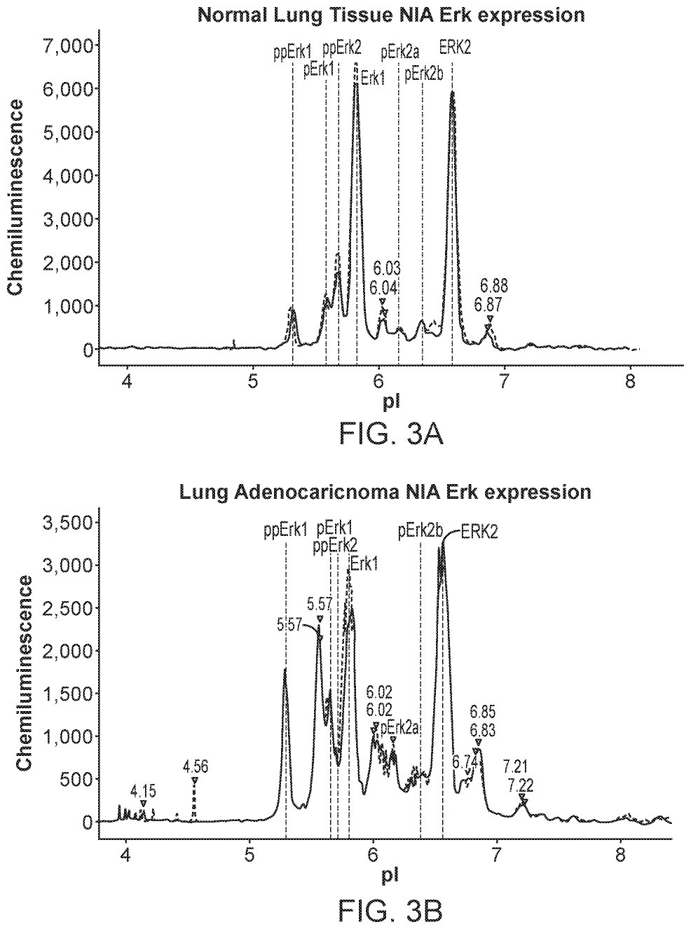

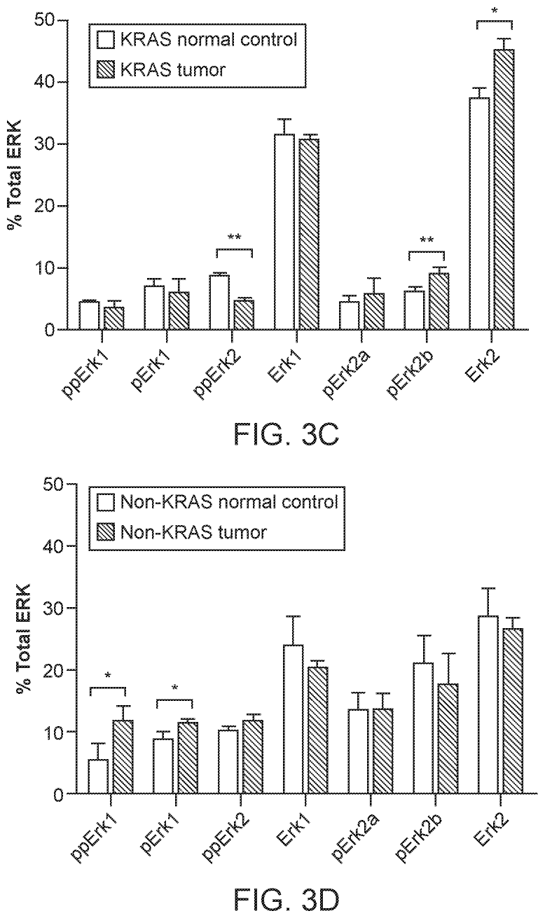

KRAS positive versus negative tumors exhibited unique ERK protein signatures. KRAS activates ERK signaling. KRAS positive versus negative lung tumors were found to exhibit ERK2 versus ERK1 activation (FIG. 3A, 3B). The KRAS positive tumors exhibited increased pERK2a, pERK2b, and ERK2 and the KRAS negative tumors exhibited increased ppERK1 and a decrease in the levels of ERK1 compared to matched normal lung tissue (FIGS. 3C and 3D). Using NIA, distinction between the different activation of various ERK phosphoisoforms by KRAS was achieved, which corroborates previous observations of ERK induction by KRAS.

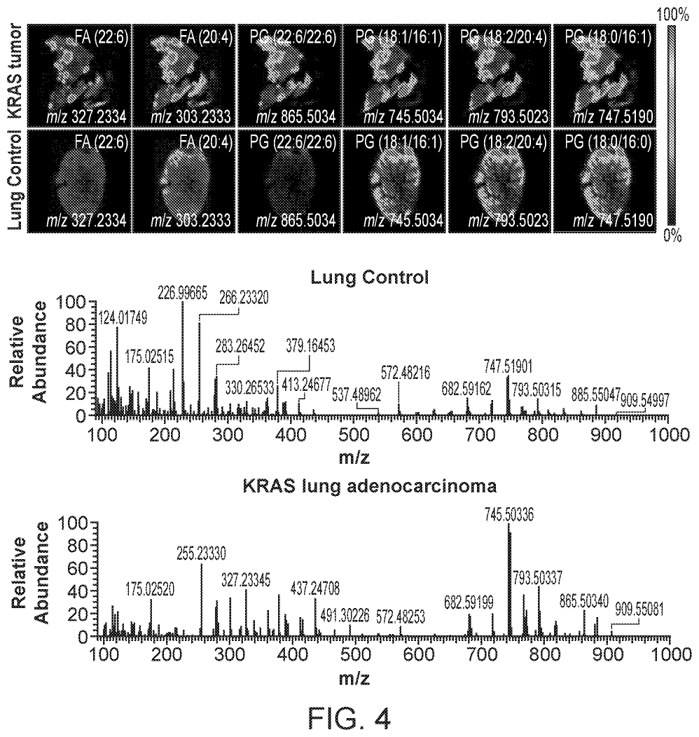

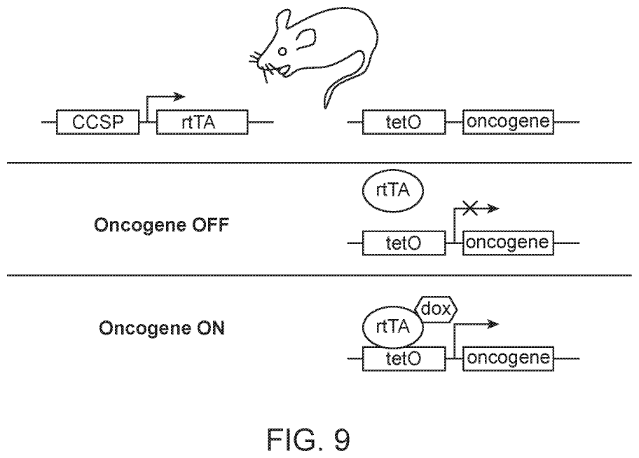

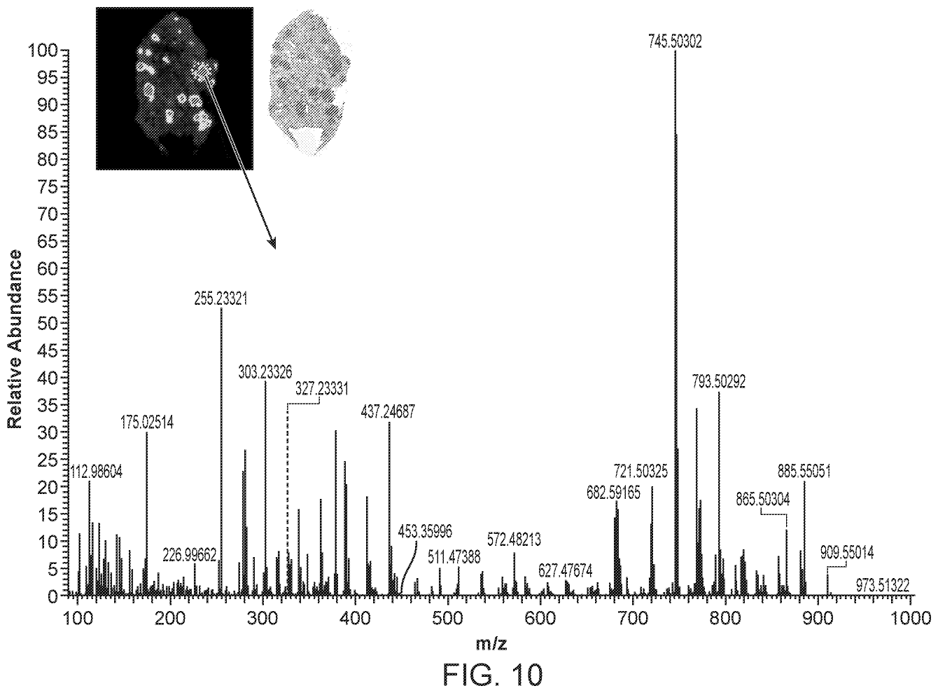

KRAS positive tumors exhibit unique lipid profiles. DESI-MSI was performed on KRAS positive mouse and human lung adenocarcinomas (see FIG. 8). Tissues were harvested from transgenic mouse models harboring a conditional KRAS activation system (see FIG. 9). We show representative mass spectra and selected two-dimensional ion images from tissue samples of KRAS-induced lung adenocarcinoma and a tissue sample of control normal lung tissue (FIG. 4, FIG. 10). As displayed in the 2D ion images of KRAS-induced lung adenocarcinoma sample (FIG. 4A), high relative intensities of lipid ions were observed in specific regions of the cancer tissue section. Histopathologic evaluation of H&E stained adjacent tissues sections confirmed that the high lipid intensity regions strongly correlated with regions of accumulation of tumor cells (FIG. 10).

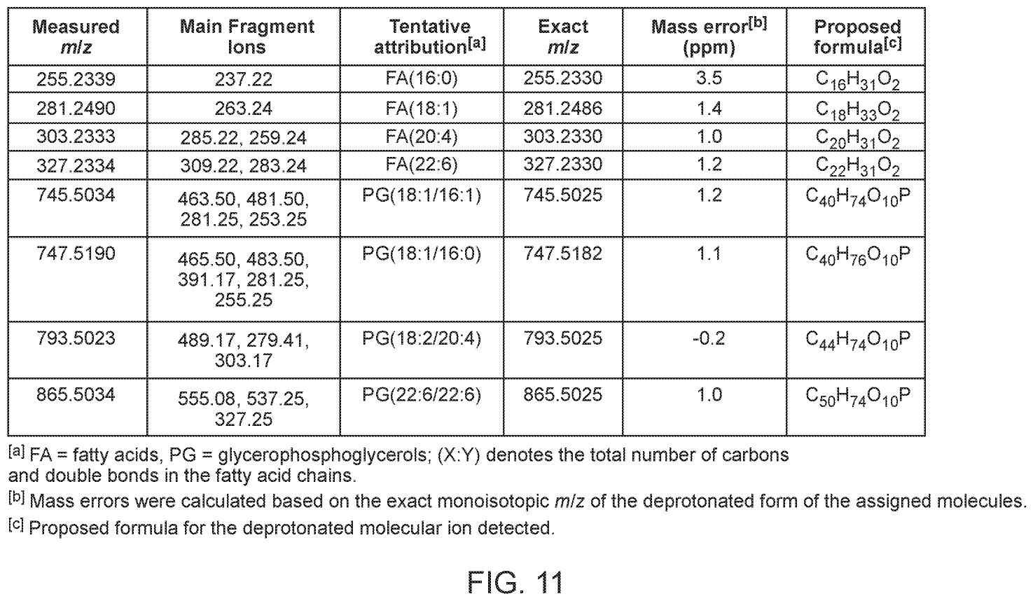

Differences in the DESI mass spectra extracted from these cancer regions were seen when compared to spectra from normal lung tissues (FIG. 4B). For example, in the m/z region 700-1000 where most complex glycerophospholipids are observed, an increase in the relative and total abundances of m/z 745.5034, PG(18:1/16:1), m/z 747.5190, as PG(18:1/16:0), m/z 793.5023, PG(18:2/20:4), and m/z 865.5034, PG(22:6/22:6), were detected. Changes in the relative and total abundances of free fatty acids, in the m/z 200-400, were also observed, including m/z 255.2339, palmitic acid FA(16:0), m/z 281.2490, oleic acid FA(18:1), m/z 303.2333, arachidonic acid FA(20:4), and m/z 327.2334, docosahexaenoic acid, FA(22:6). All identifications were made by tandem mass spectrometery (FIG. 11). While the majority of these lipid species are common in both the adjacent normal tissues and normal lung control tissues, the total and relative abundances of these species are remarkably lower in normal tissue than in the cancer tissues. These results were consistently observed in other samples of KRAS-induced lung cancer and normal lung samples from other mice. Our results suggest that KRAS induces overexpression of lipids including fatty acids and phospholipids, and is associated with a lipid profile that is distinct from normal lung tissues.

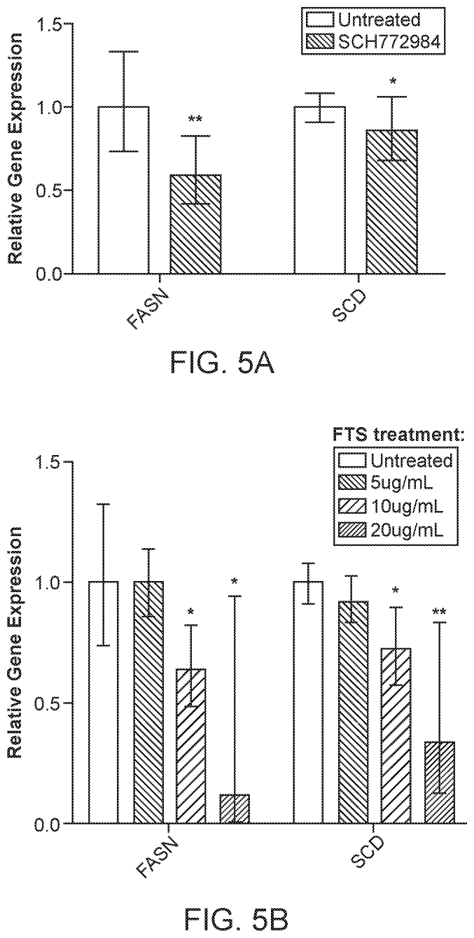

KRAS-associated induction of FASN is required for lung cancer cell proliferation. The human lung adenocarcinoma associated cell lines A549 and H1299 cells, are KRAS positive. Since we previously showed KRAS activates ERK and fatty synthesis genes, we administered the ERK inhibitor SCH772984 to both cell lines and found suppression of FASN and SCD (FIG. 5A). Moreover, KRAS inhibition using S-trans,trans-farnesylthiosalicylic acid (FTS) decreased the gene expression of FASN and SCD in a dose-dependent manner, as measured by qPCR (FIG. 5B). Therefore, KRAS inhibition impedes expression of lipogenesis associated genes. To inhibit fatty acid synthesis, we chose to inhibit FASN (FIG. 11) for several reasons. First, inhibition of SREBP would lead to suppression of various lipogenic pathways, not only fatty acid synthesis pathway. Second, inhibition of SCD would reduce the synthesis of only desaturated fatty acids, but not saturated ones. Third, FASN inhibition would be specific to fatty acid synthesis pathway which would deplete both desaturated and saturated fatty acid production.

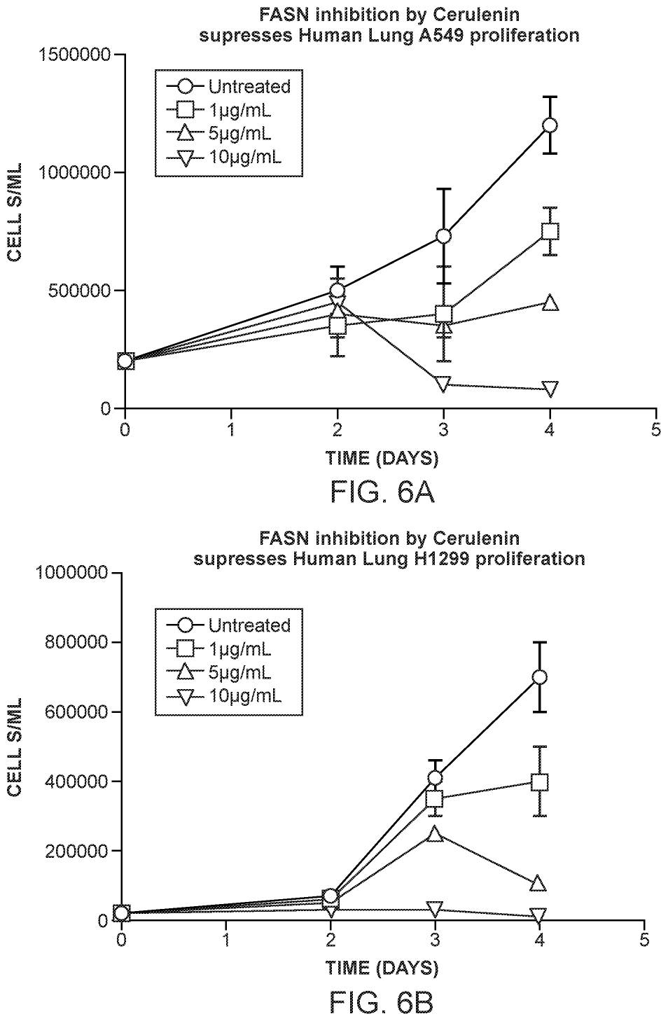

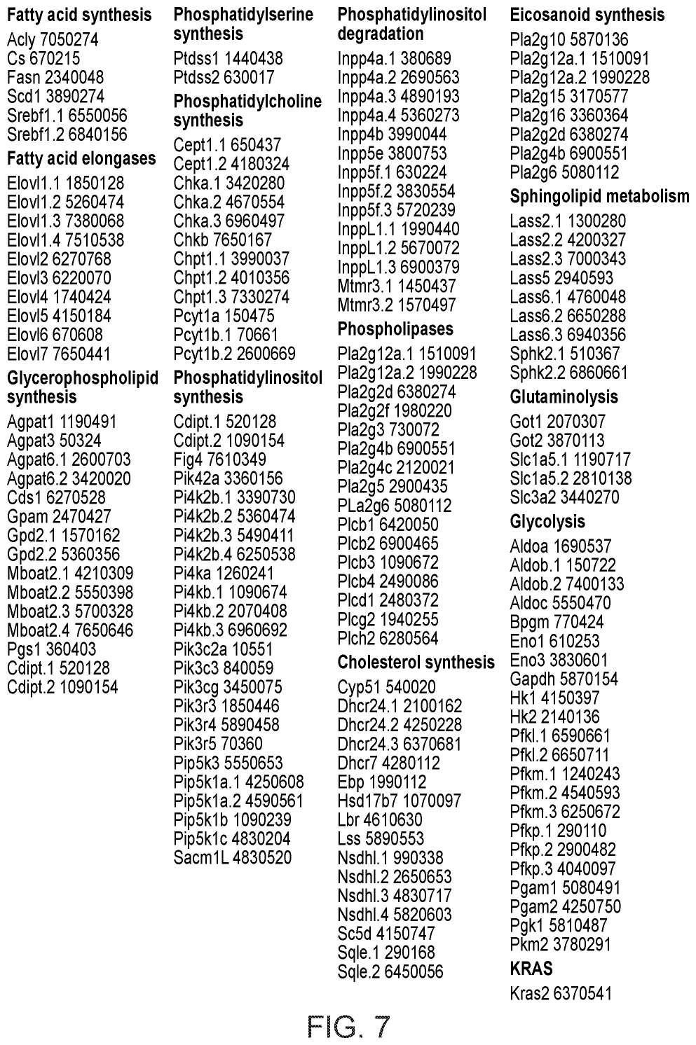

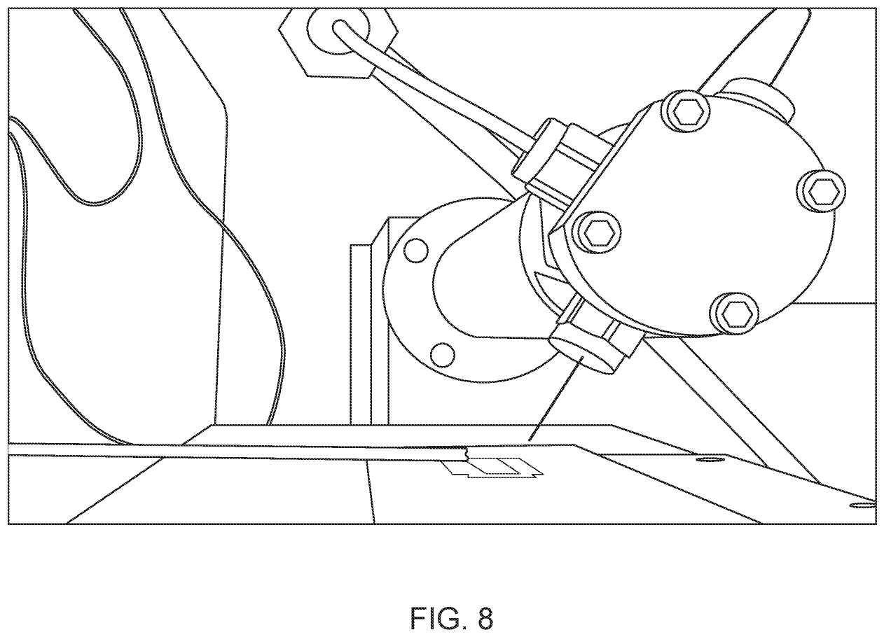

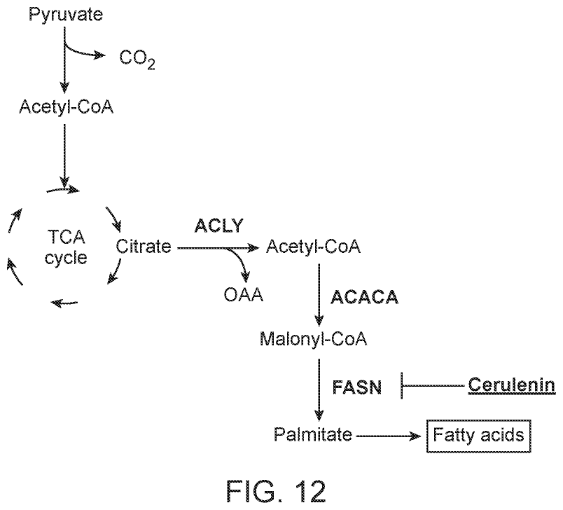

Cerulenin is an inhibitor of FASN (FIG. 12). Cerulenin treatment of mutated KRAS human lung adenocarcionma cell lines A549 and H1299 resulted in decreased proliferation as measured by PI assay and hematocytometer (FIGS. 6A and 6B). Thus, the inhibition of FASN provides a treatment for KRAS associated lung tumors.

We found that we can distinguish between KRAS positive and negative lung adenocarcinoma, as well as between neoplastic and normal lung tissue through: (a) gene expression of FASN measured by qPCR, (b) presence of ERK1 phosphoisoforms identified by the NIA, and (c) the induction of unique lipid signature detected by DESI-MSI. The inhibition of KRAS with FTS blocked FASN expression and resulted in decreased lipogesis. Moreover, the inhibition of FASN by cerulenin suppressed the proliferation of human KRAS-positive lung cancer cells. Hence, we have identified unique gene expression, as well as proteomic and lipid signature for KRAS positive lung adenocarcinoma.

Previous works demonstrate that RAS family members can regulate glucose and glutamine metabolism. Based on the present work we suggest that KRAS gene causally controls lipogenesis. KRAS signaling is well-known to generally activate the ERK and MAPK pathways. Our results show that this signaling induces lipogenesis through the induction of FASN and that it is mediated by ERK. Previous observations suggested that human tumors exhibit changes in lipid metabolism. One mechanistic basis of this is through KRAS and ERK activation.

We identified unique proteomic and lipid signatures of KRAS lung adenocarcinoma. By NIA, ERK1 versus ERK2 protein activation allowed us to distinguish between KRAS positive and negative tumors in clinical specimens. NIA is a highly sensitive nanofluidic approach that is highly tractable for the examination of even picograms of protein derived from as few as 20 cells to measure proteins and their phosphorylation state. Thus, NIA measurement of ERK may be useful in the diagnosis of lung adenocarcinoma.

Similarly, we identified a distinct lipid signature associated with KRAS positive tumors by DESI-MSI. The unprecedented ability of this technique to examine metabolic changes in situ suggests that DESI-MSI can be employed to distinguish between neoplastic and normal lung tissue, identify distinct genetic subtypes of lung cancer, and detect metabolomic alterations in lung tumors.

To date, lung adenocarcinomas appear to be incurable despite the addition of both immuno-therapeutics and EGFR receptor-targeted therapies to conventional chemotherapy. Our results suggest that KRAS-induced lung adenocarcinoma may be particularly susceptible to the targeted therapeutic inhibition of FASN.

### Materials and Methods

Desorption electrospray ionization mass spectrometry imaging (DESI-MSI). DESI-MSI was used for generating two-dimensional chemical maps of 16 μm-thick tissue sections in order to assess the lipid profiles of tissue. The lung samples were snap-frozen in liquid nitrogen and stored at −80° C. before processing. The frozen samples were cut into 16 μm sections at −21° C. using a cryomicrotome and thaw-mounted onto microscope slides. The slides were stored at −80° C. Prior to analysis, the slides were dried under a vacuum in a desiccator for approximately 20 minutes. A laboratory-built DESI-MSI source coupled to an LTQ-Orbitrap-XL mass spectrometer (Thermo Fisher Scientific) was utilized, and DESI-MSI was performed in the negative ion mode at m/z 90-1,000 with a spatial resolution of 200 μm (FIG. 7). The Orbitrap was used as the mass analyzer while set to 60,000 resolving power. Mouse tissue samples were imaged by this method using dimethylformamide and acetonitrile (1:1) as a solvent system at a flow rate of 0.5 μL/min. The N₂ pressure was set to 175 psi. In DESI-MSI, charged solvents are sprayed onto the tissue, resulting in molecules, such as metabolites and lipids, to be dissolved and extracted from the tissue surface, then transferred into a mass spectrometer for measurement of the mass-to-charge (m/z) ratios. The software ImgGenerator was used for converting raw files into 2D images. Spatially accurate ion images were assembled using BioMap software. After DESI-MSI, the same tissue section was subjected to a standard H&E staining for histopathologic evaluation using light microscopy. DESI-MS ion images were compared with optical microscopy images of the same-tissue H&E-stained tissue sections for delineation of tumor foci. Tandem MS analyses were performed using both the Orbitrap and the linear ion trap for mass analysis to confirm lipid identity. The LipidMaps database was also used to assist in lipid identification.

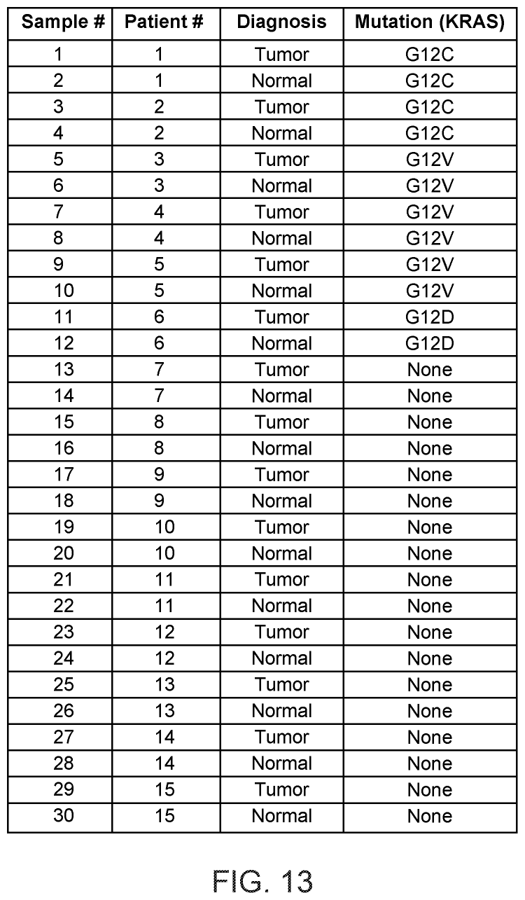

Patient tissue samples. Human lung adenocarcinoma samples with their matched normal tissue controls were acquired from the Vanderbilt Translational Pathology Shared Resource tissue bank under approved IRB protocol. The patient samples used in this study along with their KRAS mutations status is shown (see FIG. 13).

Tetracycline-based conditional murine models. A Tet-On system was used to conditionally activate KRAS4b^{G12D}, an oncogenic form of a KRAS splice variant, in the lung tissue of transgenic mice. In this system, the gene encoding a reversible tetracycline transactivating factor (rtTA) is placed under the control of the Clara cell secretory protein (CCSP) promoter that drives its constitutive expression in lung Clara cells and type II pneumocytes. KRAS4b^{G12D} cDNA is placed under the control of the tetracycline-responsive minimal promoter (TetO-KRAS4b^{G12D}). In the absence of tetracycline, rtTA binds TetO sequences and represses transcription of the oncogene. In the presence of tetracycline, the binding of doxycycline to rtTA renders rtTA unable to bind TetO sequences, resulting in the activation of oncogene transcription. Mutant KRAS is activated by administering doxycycline (Sigma) to the drinking water (100 mg/mL) starting at the age of 4 weeks. KRAS induced lung tumors from four mice and normal lung tissues from three mice were harvested at week 24 and were then analyzed by DESI-MSI. All animal studies were approved by the Stanford University Administrative Panel on Laboratory Animal Care (APLAC). CCSP-rtTA/TetO-KRAS4b^{G12D} bitransgenic mice were used. An illustration of the Tet-On regulatory system present in these transgenic mice is shown (FIG. 8).

Microarray. Microarray analyses were performed by the Stanford Functional Genomics Facility using the Illumina WG6 mouse microarray platform. Whole genome gene expression profiling was performed to compare gene expression for lung tissue from transgenic mice with KRAS turned on versus KRAS turned off. The data was log 2-transformed and quantile normalized.

Cell culture. The human non-small cell lung carcinoma cell lines, A549 and H1299, were used for in vitro experiments. A549 cells were maintained in Dulbecco's Modified Eagle Medium (DMEM) supplemented with 10% fetal bovine serum (FBS), 1% L-glutamine, 1% sodium pyruvate, 1% nonessential amino acids, and Antibiotic-Antimycotic. H1299 cells were maintained in RPMI 1640 medium supplemented with 10% FBS, 50 μM β-mercaptoethanol, and Antibiotic-Antimycotic. Trypsin-EDTA was used to passage both A549 and H1299 cells. All cell culture reagents were purchased from Gibco® (Thermo Fisher Scientific Inc.).

Small molecule inhibitors. The small molecules, SCH772984 (Cayman) at 125 ng/mL, Farnesyl Thiosalicylic Acid (FTS) (Sigma) at 5-20 ng/mL and cerulenin (Sigma) at 1-10 μg/mL, were added to cell culture medium to achieve inhibition of ERK, KRAS, and fatty acid synthase (FASN), respectively. FTS is a synthetic famesylcysteine mimetic which interferes with the anchoring of KRAS to the plasma membrane. Cerulenin irreversibly inhibits FASN by covalently binding to the enzyme. Various levels of these drugs were administered to cells in culture over a time course in order to assess dose-response relationships.

Cell counting. A volume of cells was removed from culture medium and mixed with an equal volume of 0.4% Trypan blue stain. Then 10 μL was taken out and placed into a hemocytometer for cell counting. Viable cell counts were used as a measure of cell proliferation.

RNA extraction and cDNA synthesis. Total RNA was isolated from cells and lung tissue using the QIAGEN RNeasy Mini kit. RNA quality and concentration were assessed by the NanoDrop® spectrophotometer. The RNA was reverse transcribed into cDNA using the SuperScript® III First-Strand Synthesis System (Invitrogen). All procedures were carried out according to manufacturer's protocols.

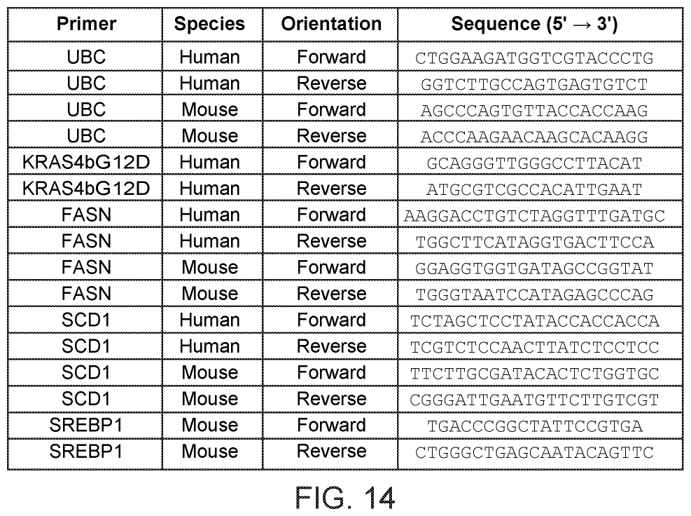

Real-time PCR. Real-time PCR was performed in 384-well plates on the QuantStudio™ 12K Flex Real-Time PCR System. Amplicons were detected by using SYBR® Green I dye as fluorophore. Reactions were carried out in 20 μL volumes that contained 1 μL cDNA, 0.5 μM forward and reverse primers, and SYBR® Green PCR Master Mix (Applied Biosystems). Amplification cycle was set as follows: 50° C. for 2 minutes; 95° C. for 10 minutes; 40 cycles of 95° C. for 15 seconds, 60° C. for 1 minute, 72° C. for 30 seconds. Following the amplification stage, a melt curve was performed to identify any non-specific amplification. For each gene, a threshold cycle (Ct) number, which represents the number of cycles required to reach the threshold fluorescence, was determined. The Ct values were exported into Excel for statistical analysis. Ubiquitin (UBC) was used as a housekeeping (reference) gene. The 2^{-ΔΔCT} method was used to determine relative mRNA expression levels. FIG. 14 lists the real-time PCR primers used in this study.

Nanoimmunoassay (NIA). NIA was performed using the Nanopro 1000 (Protein Simple) to detect the phosphorylation states of ERK1 and ERK2 in lysates generated from lung tumor tissue. NIA is a highly sensitive capillary-based isoelectric focusing method that uses antibody detection to quantify protein isoforms as well as characterize post-translational protein modifications such as phosphorylation. The final protein concentration loaded into each capillary of the Nanopro 1000 was 0.1 μg/μL. The primary rabbit antibody for ERK1/2 (Millipore) was diluted 1:300 and the primary mouse antibody for the loading control, Hsp70 (Santa Cruz Biosciences), was diluted 1:500. The anti-mouse and anti-rabbit secondary antibodies, conjugated to horse radish peroxidase, were diluted 1:100. Chemiluminescence signal was recorded following the addition of luminol and peroxide detection reagents. Analysis of NIA data was performed in Compass software (Protein Simple).

Statistical analysis. Error bars were constructed based on calculated standard deviation (SD) values. The error bars represent the mean±SDs. Where appropriate, a student's t-test was used to assess statistical significance. Statistical significance by t-test is indicated by * for p-value<0.05; ** for p-value<0.01; *** for p-value<0.001.

### Example 2

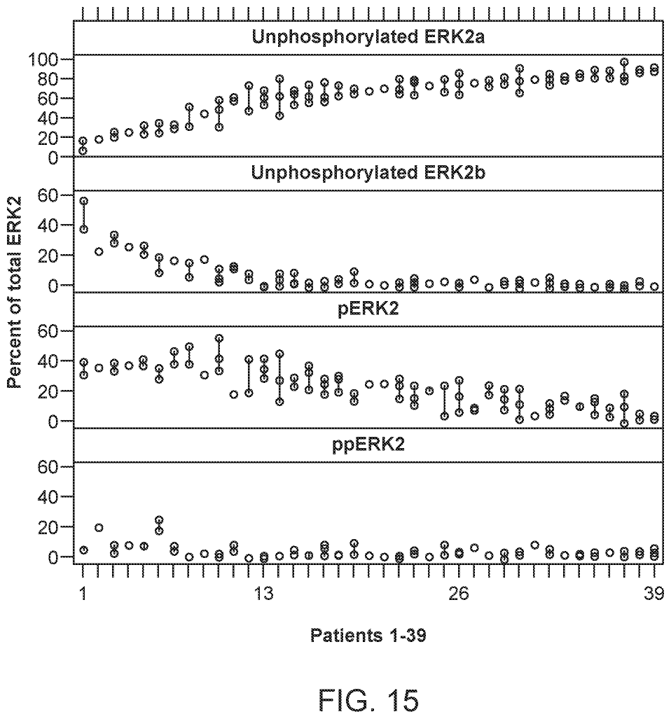

Nanoscale Protein measurements can be used to assess intratumoral vs intrapatient heterogeneity of solid tumors. Nano-immunoassay (NIA) was used to analyze ERK signaling in fine needle aspirate biopsies (FNA's) from patients with kidney cancer. 39 patients each had 2-3 regions of their kidney tumor sampled by FNA. Shown in FIG. 15, each circle is a tumor FNA (N=91 FNAs), averaged across technical replicates. Samples from each patient are connected by a vertical line. Patients are ordered by the average across samples of ERK2. Across isoforms, the variation across technical replicates has an average standard deviation of 1%.

In samples from different regions of the same tumor, the variation between samples has an average standard deviation of 6%. In contrast, the standard deviation of the measured proportions across different patients ranges from 5% to 22% across isoforms.

These data demonstrate that for ERK, a key signaling protein, intratumoral heterogeneity (6%) is less than intrapatient heterogeneity (22% for phospho-isoform pERK2b). This finding demonstrates that analysis of ERK isoforms in a single FNA from a tumor can be representative of the whole tumor.

### Example 3

NIA can be Used to Measure Proteins in Clinical Specimens from Patients with Renal Cell Carcinoma (RCC) and Lung Cancer

As described herein, NIA charge separation has been used to measure new proteins in many specimen types and different malignancies. For example, specimen types include, but are not limited to flash frozen tumor, frozen sections embedded in OTC, fine needle aspirates, bone marrow, blood, CTCs, and plasma. Human tumor clinical specimens include, but are not limited to lymphoma, CML, MDS, kidney cancer, lung cancer, and head and neck cancer. Drugs used include, but are not limited to atorvastatin, rigosertib, FTS, and anti-EPHA3. The data presented herein demonstrate that NIA can be used for diagnostics. For kidney cancer, glutaminase levels are measured. For lung cancer, RAS⁺ vs RAS⁻ are distinguished. Also, different types of cancer are distinguished from one another, such as between kidney, head, and neck cancer. The data presented demonstrates that NIA can be used for therapeutic monitoring and drug development. Drugs such as FTS (preclinical), Rigosertib (clinical trial), and Atorvastatin (clinical trial) have been used.

The data presented herein illustrates that NIA can be used as diagnostic tool, as seen through the ability of NIA signaling measurements to distinguish between different tumor types, the ability of NIA to measure ERK signaling in CTCs from lung cancer patients, and the ability of NIA to differentiate KRAS+ from KRAS− lung tumors. Furthermore, the data presented herein illustrates that NIA can monitor therapeutic efficacy, as seen through monitoring ERK levels in FTS-treated tumors, and NIA differentiation pre vs. post Rigosertib treated head and neck cancer (HNSCC).

NIA can be used to measure proteins in clinical specimens from patients with RCC and Lung Cancer.

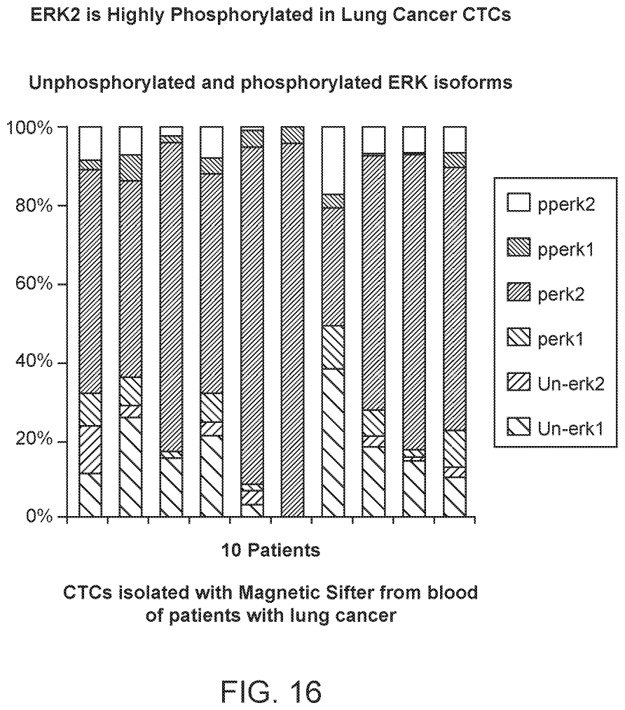

As shown in FIG. 16, circulating tumor cells were isolated from 10 patients using Mag-Sifter technology: cells from each patient were frozen in pellets. Cell pellets from each patient were analyzed using NIA to measure ERK isoforms.

### Example 4

### NIA Detects Changes by FTS Inhibition of RAS

The data presented herein illustrates that NIA can monitor therapeutic efficacy, as seen through monitoring Akt and ERK levels in FTS-treated tumors, and NIA differentiation pre vs. post Rigosertib treated head and neck cancer (HNSCC).

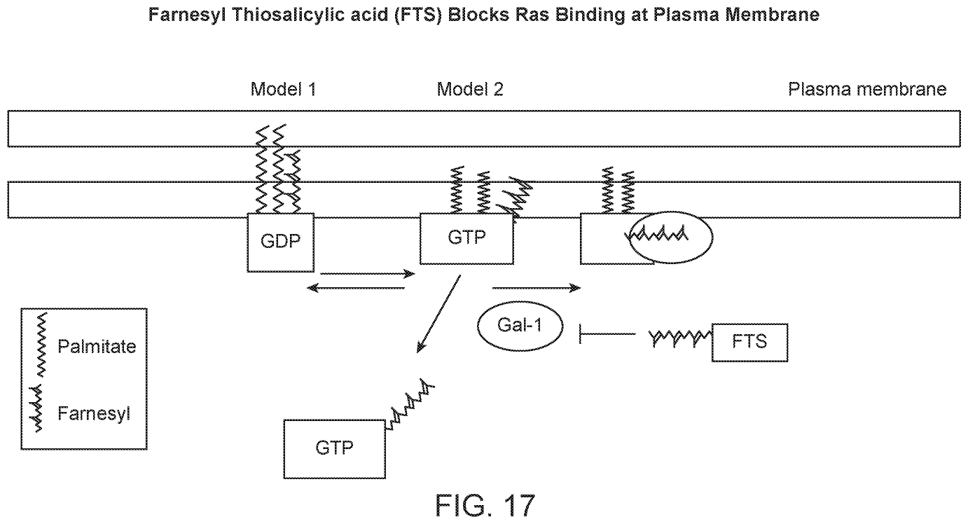

NIA detects changes by FTS inhibition of RAS. Farnesyl Thiosalicylic acid (FTS) Blocks Ras Binding at Plasma Membrane, as shown in FIG. 17.

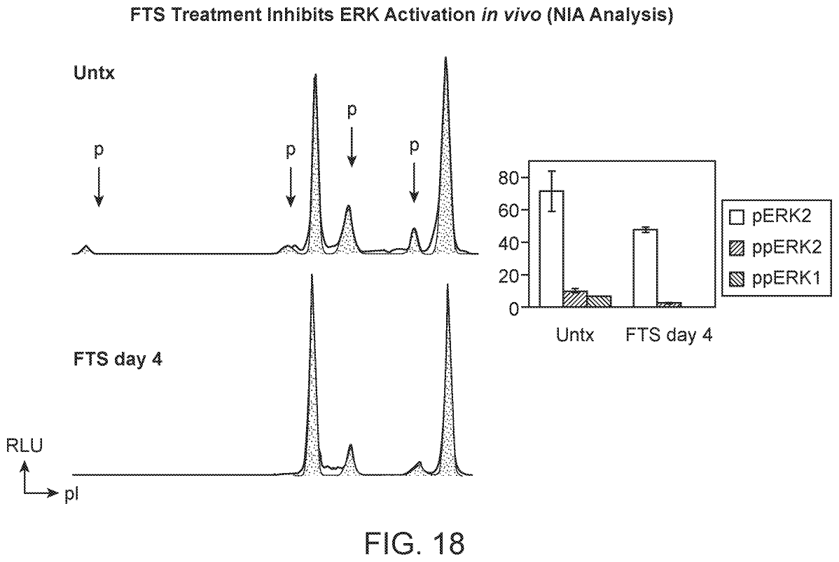

As shown in FIG. 18, FTS treatment Inhibits ERK Activation in vivo (NIA Analysis). Mice with MYC-induced lymphoma were treated with FTS. Tumors from mouse untreated or after 4 days of treatment were sampled by fine needle aspirate. FNAs were flash frozen and batch analyzed. NIA was used to analyze ERK isoforms using NIA. Total ERK1/2 Level did not change, whereas all phospho-ERK isoforms decreased on day 4.

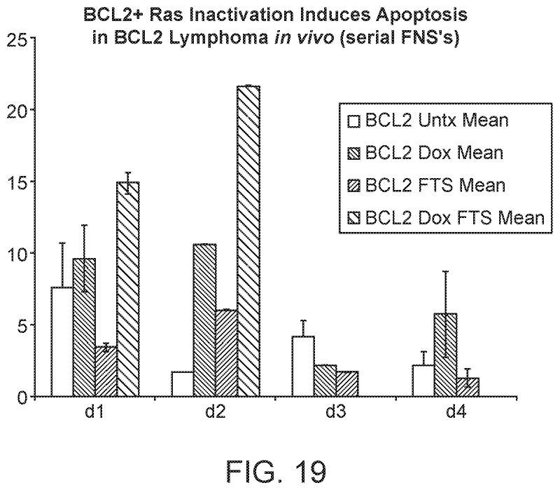

As shown in FIG. 19, BCL2+ Ras inactivation induces apoptosis in BCL2 lymphoma in vivo (serial FNA's). Mice with BCL2-induced lymphoma were untreated (BCL2 Untx), treated to inactivate BCL2 (BCL2 Dox), treated with FTS alone to inhibit Ras (BCL2 FTS) or treated to inactivate both BCL2 and Ras (BCL2 Dox FTS). Tumors from mouse untreated or after 1, 2,3, or 4 days of treatment were sampled by fine needle aspirate. FNAs were flash frozen and batch analyzed. NIA was used to analyze ERK isoforms using NIA.

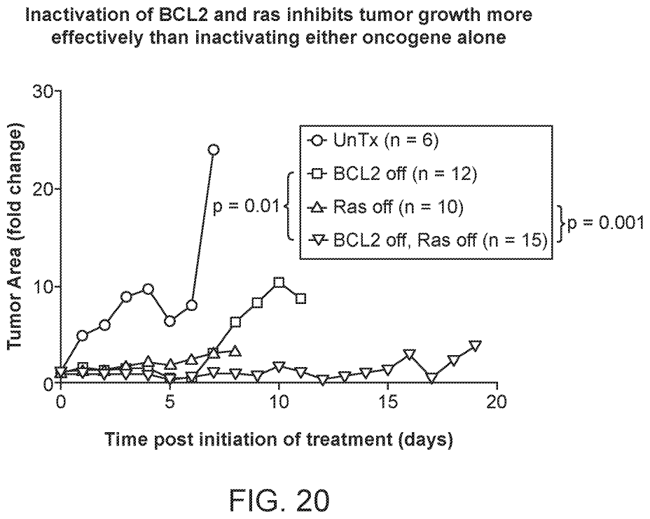

FIG. 20 shows inactivation of BCL2 and ras inhibits tumor growth more effectively than inactivating either oncogene alone. Inactivation of both BCL2 and Ras inhibits tumor growth more effectively than either oncogene alone: mice with BCL2-induced lymphoma. Plots of tumor area over time in mice cohorts: untreated (UnTx), with BCL2 inactivated (BCL2 off), Ras inactivated (Ras off, or both BCL2 and Ras inactivated (BCL2off,Ras off).

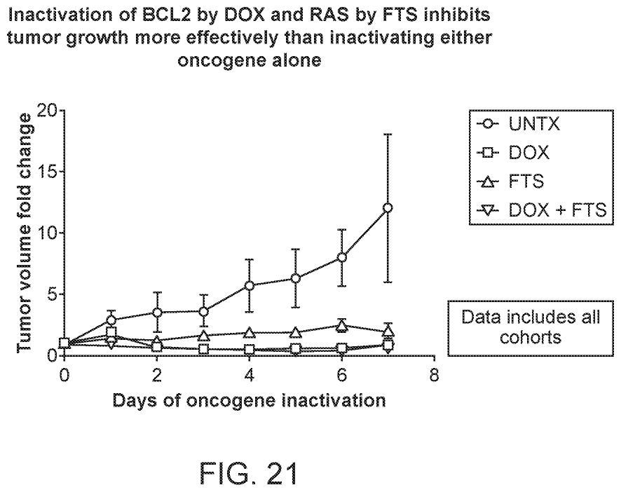

As shown in FIG. 21, inactivation of BCL2 by DOX and RAS by FTS inhibits tumor growth more effectively than inactivating either oncogene alone.

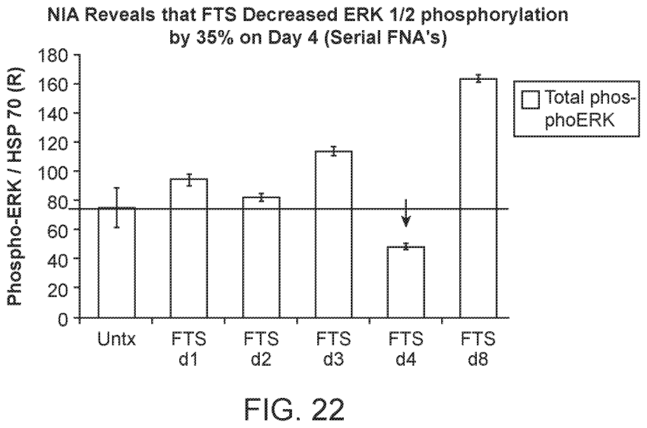

FIG. 22 shows NIA analysis of ERK isoforms in FNAs from transgenic lymphoma. NIA reveals that FTS decreased ERK1/2 phosphorylation by 35% on Day 4. Tumors were sampled by fine needle aspirate prior to treatment (UnTx), and treated after 1-8 days of treatment with FTS.

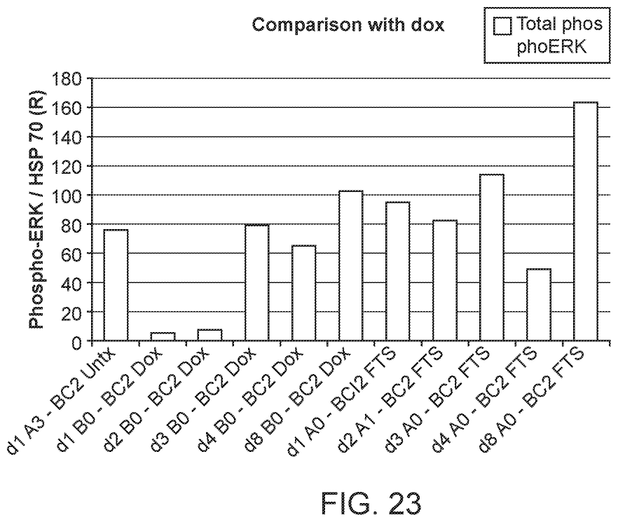

FIG. 23 shows NIA analysis of ERK isoforms in FNAs from transgenic lymphoma. Comparison with dox is shown. Mice were either treated with FTS (BC2FTS) or BCL2 inactivation (BCL2Dox). Tumors were sampled by fine needle aspirate prior to treatment (UnTx), and treated after 1-8 days of treatment with FTS.

### Example 5

### NIA Detects Changes by Atorvastatin Inhibition of HMG-CoA Reductase

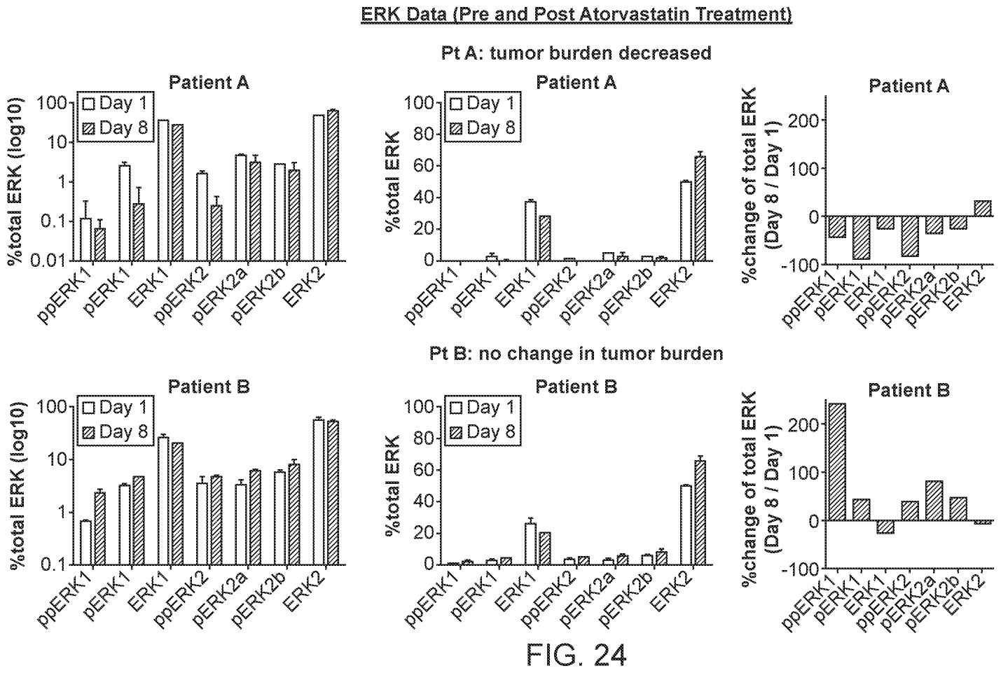

FIG. 24 shows ERK Data (pre and post Atorvastatin treatment). Two patients were treated on clinical trial of HMG co-A reductase inhibitor atorvastatin. NIA was used to measure changes in ERK isoforms in tumor cells sampled before and after eight days of treatment. For patient A, tumor burden decreased. For patient B, no change in tumor burden was seen.

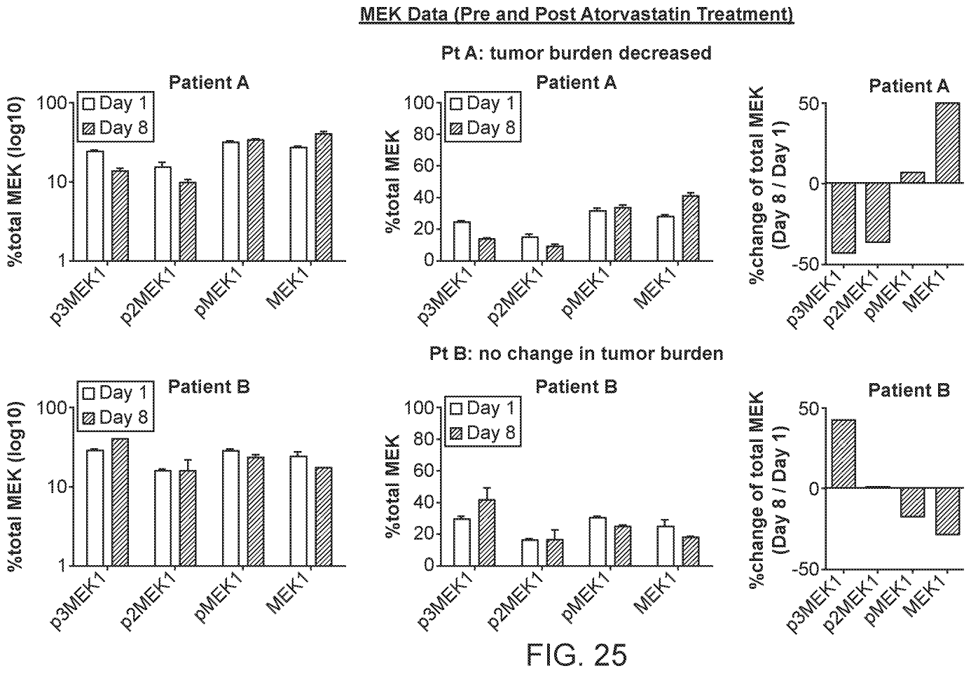

FIG. 25 shows MEK data (pre and post atorvastatin treatment). Two patients were treated on clinical trial of HMG co-A reductase inhibitor atorvastatin. NIA was used to measure changes in MEK isoforms before and after eight days of treatment. For patient A, tumor burden decreased. For patient B, no change in tumor burden was seen.

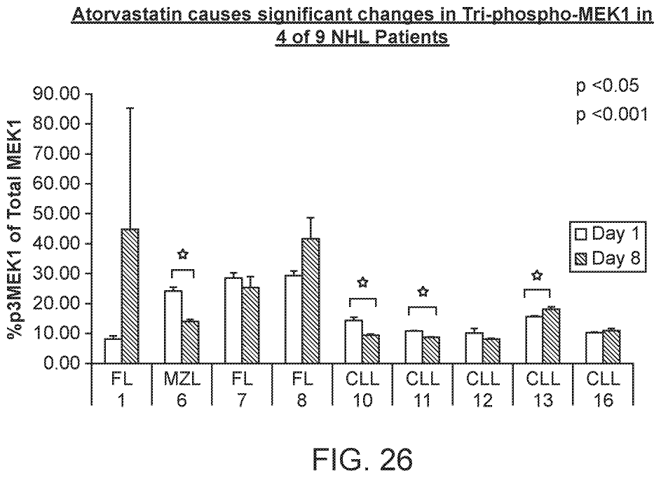

As shown in FIG. 26, atorvastatin causes significant changes in tri-phospho-MEK1 in four of nine NHL patients. Nine patients were treated on clinical trial of HMG co-A reductase inhibitor atorvastatin. NIA was used to measure changes in MEK isoforms before (day 1) and after eight days of treatment.

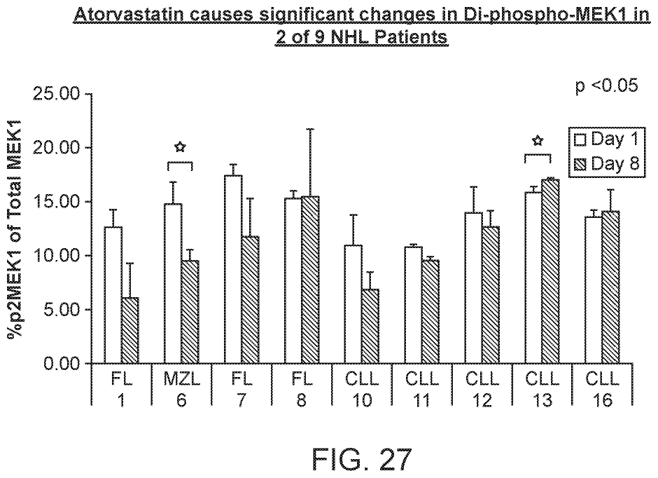

FIG. 27 shows atorvastatin causes significant changes in di-phospho-MEK1 in four of nine NHL patients. Nine patients were treated on clinical trial of HMG co-A reductase inhibitor atorvastatin. NIA was used to measure changes in MEK isoforms before (day 1) and after 8 days of treatment.

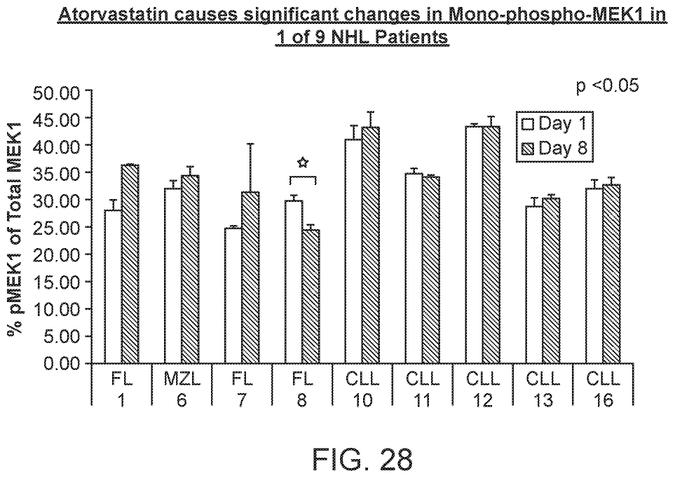

FIG. 28 shows atorvastatin causes significant changes in Mono-phospho-MEK1 in one of nine NHL patients. Nine patients were treated on clinical trial of HMG co-A reductase inhibitor atorvastatin. NIA was used to measure changes in MEK isoforms before (day 1) and after eight days of treatment.

### Example 6

### NIA Detects Changes by Rigosertib Inhibition of PI3K

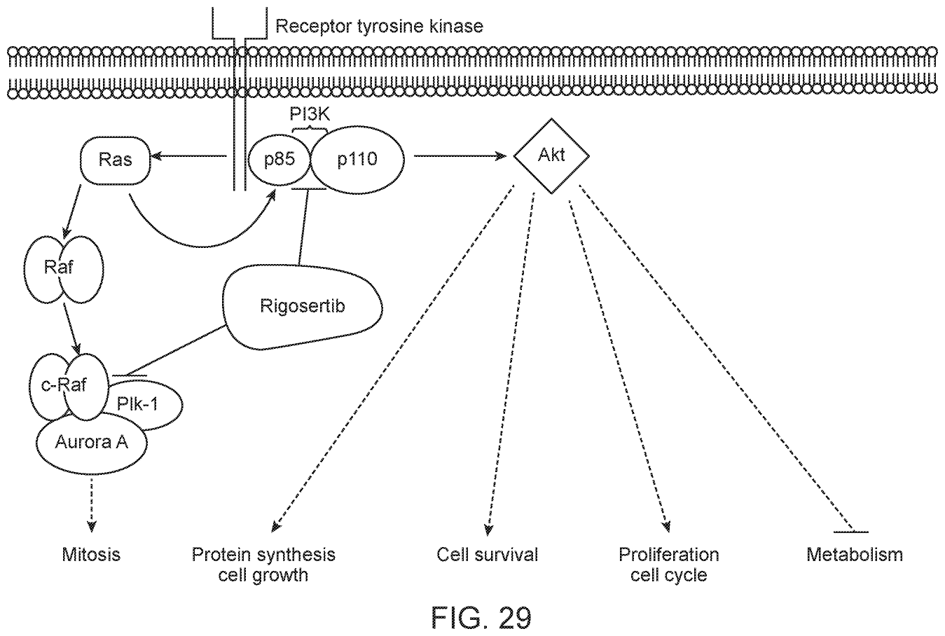

Erk signature as therapeutic assessment by NIA now in solid tumors was reviewed. Erk activity by NIA as potential biomarker for therapeutic response to Rigosertib treatment in patients with Platinum-resistant, relapsed or metastatic H&N SCC was assessed, as illustrated in FIG. 29.

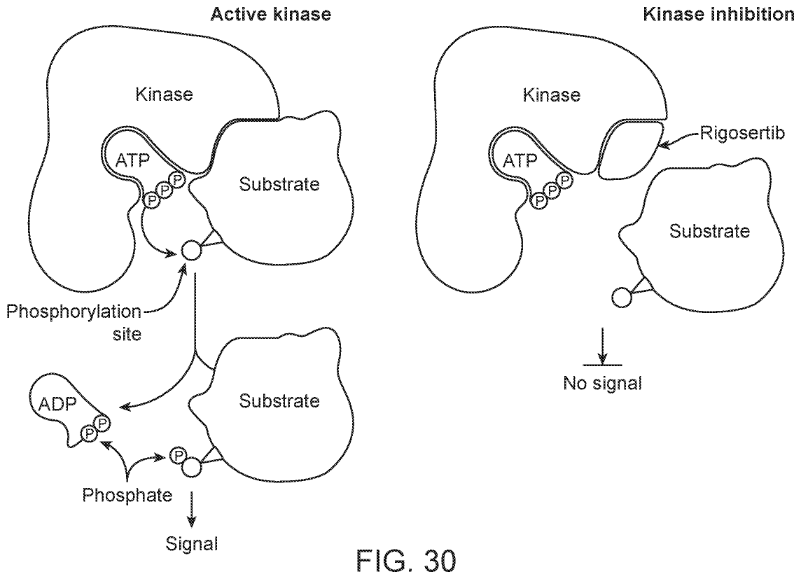

FIG. 30 shows Rigosertib's mechanism of action. Rigosertib is an allosteric inhibitor of protein kinase activity. It is known to inhibit both the PI3K and Plk1 pathways in CML and MDS.

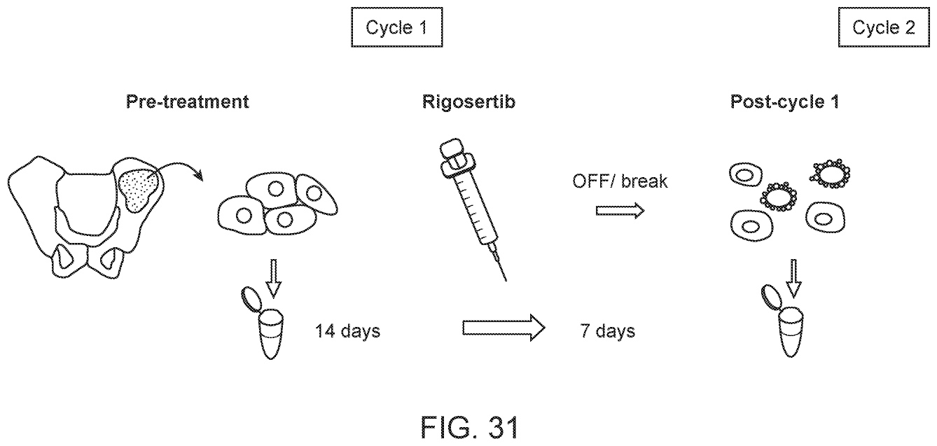

As shown in FIG. 31, for Rigosertib treatment, patients are administered 560 mg BID Rigosertib, for 14 consecutive days of a 21-day cycle (2 weeks on, 1 week off regimen). Patients receive 2 cycles of 2 weeks of treatment with 1 week off regimen. For Cycle 1, Day 14 only, 5 patients with HNSCC will undergo biopsy or FNA for nano-proteomic profiling of total- and phospho-proteins in pathways inhibited by rigosertib, ERK and loading control. For Cycle 1 Day 14 and Cycle 2 Day 14, pharmacokinetic blood analyses are performed.

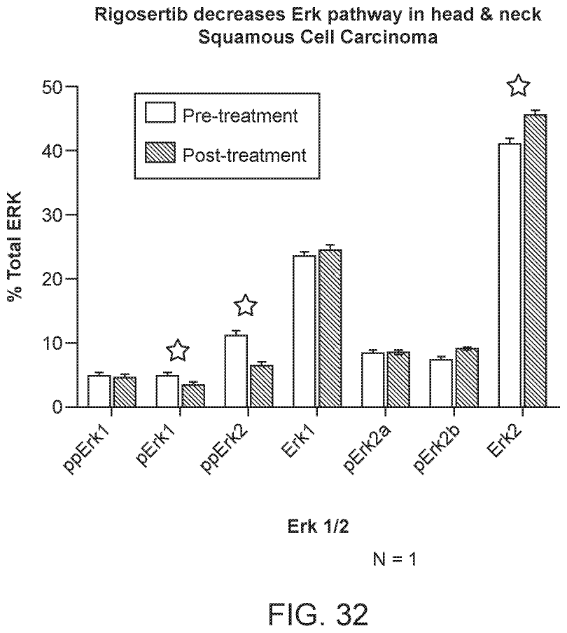

FIG. 32 shows that Rigosertib decreases Erk pathway in head & neck squamous cell carcinoma. A patient with head and neck cancer treated on clinical trial of rigosertib had tumor sampled before (pre-treatment) and after (post-treatment) treatment. NIA was used to measure ERK isforms.

All publications mentioned herein are incorporated herein by reference for the purpose of describing and disclosing, for example, the compounds and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

What is claimed is:

1. A method of determining if a tumor of a patient is driven by a KRAS mutation (KRAS⁺), the method comprising:
obtaining a sample of a tumor suspected of being KRAS⁺; and
performing one or both of:
a nanofluidic proteomic immunoassay (NIA) for ERK phosphoisoforms; and
desorption electrospray ionization mass spectrometry imaging (DESI-MSI) for lipid species in the region of from about m/z region 700-1000 and/or about m/z 200-400;
determining whether the sample displays altered ERK1 isoforms and/or altered lipid species relative to a KRAS⁻ tumor or normal tissue; wherein a KRAS⁺ tumor displays altered ERK1 isoforms and/or altered lipid species relative to a KRAS⁻ tumor or normal tissue; and
providing the determination to the patient.

2. The method of claim 1, further comprising treating the patient in accordance with the determination.

3. The method of any of claims 1-2, wherein the tumor is a lung adenocarcinoma.

4. The method of any of claims 1-3, wherein the sample is a biopsy sample.

5. The method of claim 4, wherein the biopsy sample is a tumor cell sample of less than 100,000 cells.

6. The method of claim 4, wherein the biopsy sample is a fine needle aspirate sample.

7. The method of claim 4, wherein the control tissue is a sample from the same tumor at a different time point.

8. The method of claim 7, wherein multiple time points from a single tumor are compared.

9. The method of claim 1, wherein the cellular sample was previously frozen.

10. The method of any of claims 1-9, wherein the NIA detects significantly increased levels of ppERK1 and pERK1 when compared to total ERK protein levels for a KRAS⁺ tumor.

11. The method of any of claims 1-10, wherein the DESI-MSI detects significantly increased levels of complex glycerophospholipids and free fatty acids for a KRAS⁺ tumor.

12. The method of claim 2, wherein the patient is treated with an inhibitor of fatty acid synthase (FASN).

13. The method of claim 12, wherein the inhibitor is administered in combination with a second therapeutic regimen.

14. The method of claim 12 or claim 13, wherein the inhibitor of FASN is cerulenin.

15. The method of claim 2, further comprising determining whether a sample from the patient displays altered ERK1 isoforms and/or altered lipid species relative to a KRAS⁻ tumor or normal tissue at two or more time points over the course of treatment to determine the effectiveness of therapy with respect to markers indicative of KRAS-driven tumorigenesis.

16. A method for identifying a subject with a KRAS⁺ cancer, the method comprising:
performing a nanofluidic proteomic immunoassay (NIA) and/or a desorption electrospray ionization mass spectrometry imaging (DESI-MSI) on a clinical sample obtained from a subject; and
measuring ERK1 phosphoisoforms and/or lipid species in the clinical sample.

17. The method of claim 16, wherein the clinical sample has significantly increased levels of ppERK1 and pERK1 when compared to total ERK protein levels.

18. The method of claim 16 or 17, wherein the clinical sample has significantly increased levels of ppERK1 and pERK1 when compared to a normal tissue sample or KRAS-cancer.

19. The method of any one of claims 16-18, wherein performing the DESI-MSI involves detecting lipid species in a region ranging from about m/z region 700-1000 and/or about m/z 200-400.

20. The method of any one of claims 16-19, wherein the clinical sample displays altered lipid species relative to a normal tissue sample or KRAS⁻ cancer.

21. The method of any one of claims 16-20, wherein the clinical sample has increased relative and/or total abundances of m/z 745.5034, PG(18:1/16:1), m/z 747.5190, as PG(18:1/16:0), m/z 793.5023, PG(18:2/20:4), and m/z 865.5034, PG(22:6/22:6).

22. The method of any one of claims 16-21, wherein the DESI-MSI detects significantly increased levels of complex glycerophospholipids and free fatty acids for a KRAS⁺ tumor.

23. The method of any one of claims 16-22, wherein the clinical sample is a blood sample.

24. The method of any one of claims 16-23, wherein the clinical sample is a biopsy sample.

25. The method of claim 24, wherein the biopsy sample is obtained from a tumor.

26. The method of any one of claims 16-25, wherein the clinical sample comprises less than 100,000 cells.

27. The method of any one of claims 16-25, wherein the clinical sample comprises less than 1,000 cells.

28. The method of any one of claims 16-25, wherein the clinical sample comprises less than 100 cells.

29. The method of any one of claims 16-28, wherein the clinical sample is obtained by fine needle aspiration.

30. The method of any one of claims 16-29, wherein the clinical sample is a fine needle aspirate (FNA) that is sampled in vivo.

31. The method of claim 30, further comprising comparing the FNA with an adjacent non-tumor tissue.

32. The method of any one of claims 16-31, wherein the subject is diagnosed with lung adenocarcinoma.

33. The method of any one of claims 16-31, wherein the subject is diagnosed with kidney cancer.

34. The method of any one of claims 16-33, further comprising performing a second NIA and/or a second DESI-MSI from the same tumor at a different time point.

35. The method of any one of claims 16-34, wherein the clinical sample was previously frozen.

36. The method of any one of claims 16-35, wherein the clinical sample was previously maintained on ice for greater than 30 minutes prior to performing the NIA and/or the DESI-MSI.

37. A method of treating or reducing a KRAS⁺ cancer in a subject in need thereof, the method comprising:
performing a nanofluidic proteomic immunoassay (NIA) and/or a desorption electrospray ionization mass spectrometry imaging (DESI-MSI) on a clinical sample obtained from a location on the subject;
measuring ERK1 phosphoisoforms and/or lipid species in the clinical sample at a first time point;
performing a second NIA and/or a second DESI-MSI on the clinical sample obtained from approximately the same location on the subject after the subject has been treated with an effective amount of an anti-cancer agent; and
measuring ERK1 phosphoisoforms and/or lipid species in the clinical sample obtained from approximately the same location on the subject after the subject has been treated with an anti-cancer agent at a second time point.

38. The method of claim 37, wherein the anti-cancer agent is a fatty acid synthase inhibitor.

39. The method of claim 37, wherein the anti-cancer agent is lipogenesis enzyme inhibitor.

40. The method of any one of claims 37-39, further comprising placing the patient on a treatment regimen, wherein the treatment regimen comprises administering an effective amount of an anti-cancer therapeutic for at least 1 month.

41. The method of any one of claims 37-40, further comprising maintaining, adjusting, or stopping the treatment regimen based on the ERK1 phosphoisoforms and/or the lipid species in the clinical sample obtained from approximately the same location on the subject after the subject has been treated with the anti-cancer agent, wherein a change in the ERK1 phosphoisoforms and/or the lipid species indicates a response to the treatment regimen.

42. The method of any one of claims 37-41, wherein the clinical sample has significantly increased levels of ppERK1 and pERK1 when compared to total ERK protein levels at the first time point.

43. The method of any one of claims 37-41, wherein the clinical sample has significantly increased levels of ppERK1 and pERK1 when compared to a normal tissue sample or KRAS⁻ cancer at the first time point.

44. The method of any one of claims 37-43, wherein the levels of ppERK1 and pERK1 are greater at the first time point than the second time point.

45. The method of claim any one of claims 37-44, wherein performing the DESI-MSI involves detecting lipid species in a region ranging from about m/z region 700-1000 and/or about m/z 200-400.

46. The method of claim any one of claims 37-45, wherein the clinical sample displays altered lipid species relative to a normal tissue sample or KRAS⁻ cancer at the first time point.

47. The method of any one of claims 37-46, wherein the clinical sample has increased relative and/or total abundances of m/z 745.5034, PG(18:1/16:1), m/z 747.5190, as PG(18:1/16:0), m/z 793.5023, PG(18:2/20:4), and m/z 865.5034, PG(22:6/22:6) at the first time point.

48. The method of claim any one of claims 37-47, wherein the DESI-MSI detects significantly increased levels of complex glycerophospholipids and free fatty acids for a KRAS⁺ tumor at the first time point.

49. The method of any one of claims 37-48, wherein the levels of complex glycerophospholipids and free fatty acids are greater at the first time point than the second time point.

50. The method of any one of claims 37-49, wherein the clinical sample is a blood sample.

51. The method of any one of claims 37-49, wherein the clinical sample is a biopsy sample.

52. The method of claim 51, wherein the biopsy sample is obtained from a tumor.

53. The method of any one of claims 37-52, wherein the clinical sample comprises less than 100,000 cells.

54. The method of any one of claims 37-53, wherein the clinical sample comprises less than 1,000 cells.

55. The method of any one of claims 37-53, wherein the clinical sample comprises less than 100 cells.

56. The method of any one of claims 37-55, wherein the clinical sample is obtained by fine needle aspiration.

57. The method of any one of claims 37-56, wherein the clinical sample is a fine needle aspirate (FNA) that is sampled in vivo.

58. The method of claim 57, further comprising comparing the FNA with an adjacent non-tumor tissue.

59. The method of any one of claims 37-58, wherein the subject is diagnosed with lung adenocarcinoma.

60. The method of any one of claims 37-58, wherein the subject is diagnosed with kidney cancer.

61. The method of any one of claims 37-60, wherein the clinical sample was previously frozen.

62. The method of any one of claims 37-61, wherein the clinical sample was previously on ice for greater than 30 minutes prior to performing the NIA and/or the DESI-MSI.

63. The method of any one of claims 37-62, wherein the subject is human.

64. The method of any one of claims 37-62, wherein the subject is an animal.

65. The method of claim 64, wherein the animal is a mouse.

66. The method of claim 64 or 65, further comprising transplanting cancer cells into the animal.

67. A method for treating a disease or disorder in a subject, the method comprising administering to the subject an effective amount of an anti-cancer agent, wherein treatment with the anti-cancer agent is based upon the levels of ppERK1 and pERK1, and/or levels of complex glycerophospholipids and free fatty acids in a clinical sample obtained from the subject, and wherein the levels of ppERK1 and pERK1, and/or levels of complex glycerophospholipids and free fatty acids are elevated in comparison to reference levels.

68. The method of claim 67, wherein the levels of ppERK1 and pERK1 are measured by nanofluidic proteomic immunoassay (NIA).

69. The method of claim 67 or 68, wherein the levels of complex glycerophospholipids and free fatty acids is measured by desorption electrospray ionization mass spectrometry imaging (DESI-MSI), wherein the DESI-MSI involves detecting lipid species in a region ranging from about m/z region 700-1000 and/or about m/z 200-400.

70. The method of any one of claims 67-69, wherein the disease or disorder is a KRAS⁺ cancer.

71. The method of claim 70, wherein the KRAS⁺ cancer is lung carcinoma.

72. The method of claim 70, wherein the KRAS⁺ cancer is kidney cancer.

73. The method of any one of claims 67-72, wherein the anti-cancer agent is a fatty acid synthase inhibitor.

74. The method of any one of claims 67-73, wherein the anti-cancer agent is lipogenesis enzyme inhibitor.

75. The method of any one of claims 67-74, wherein the reference levels are the levels of ppERK1 and pERK1, and/or levels of complex glycerophospholipids and free fatty acids in a KRAS⁻ tumor or normal tissue.

76. The method of claim any one of claims 67-75, wherein the clinical sample is a blood sample.

77. The method of claim any one of claims 67-75, wherein the clinical sample is a biopsy sample.

78. The method of claim 77, wherein the biopsy sample is obtained from a tumor.

79. The method of any one of claims 67-78, wherein the clinical sample comprises less than 100,000 cells.

80. The method of any one of claims 67-78, wherein the clinical sample comprises less than 1,000 cells.

81. The method of any one of claims 67-78, wherein the clinical sample comprises less than 100 cells.

82. The method of any one of claims 67-81, wherein the clinical sample is obtained by fine needle aspiration.

83. The method of any one of claims 67-82, wherein the clinical sample was previously frozen.

84. The method of any one of claims 67-83, wherein the clinical sample was previously on ice for greater than 30 minutes prior to performing the NIA and/or the DESI-MSI.

85. A method of treating or reducing cancer a KRAS⁺ cancer in a subject in need thereof, the method comprising:
transplanting cancer cells into a location of an animal;
removing a portion of the cancer cells from the location;
treating ex vivo the portion with an effective amount of an anti-cancer agent to generate a treated portion;
performing a nanofluidic proteomic immunoassay (NIA) and/or a desorption electrospray ionization mass spectrometry imaging (DESI-MSI) on the treated portion; and
measuring ERK1 phosphoisoforms and/or lipid species in the portion.

86. The method of claim 85, wherein the animal is a mouse.

87. The method of claim 85 or 86, further comprising performing a nanofluidic proteomic immunoassay (NIA) and/or a desorption electrospray ionization mass spectrometry imaging (DESI-MSI) on the portion; and measuring ERK1 phosphoisoforms and/or lipid species in the portion prior to treating with the anti-cancer agent.


SEQUENCE LISTING (16 sequences)

SEQ ID NO: 1 - DNA, 21 bp, Homo Sapiens
ctggaagatggtcgtaccctg

SEQ ID NO: 2 - DNA, 20 bp, Homo Sapiens
ggtcttgccagtgagtgtct

SEQ ID NO: 3 - DNA, 20 bp, Mus musculus
agcccagtgttaccaccaag

SEQ ID NO: 4 - DNA, 20 bp, Mus musculus
acccaagaacaagcacaagg

SEQ ID NO: 5 - DNA, 19 bp, Homo Sapiens
gcagggttgggccttacat

SEQ ID NO: 6 - DNA, 19 bp, Homo Sapiens
atgcgtcgccacattgaat

SEQ ID NO: 7 - DNA, 23 bp, Homo Sapiens
aaggacctgtctaggtttgatgc

SEQ ID NO: 8 - DNA, 21 bp, Homo Sapiens
tggcttcataggtgacttcca

SEQ ID NO: 9 - DNA, 21 bp, Mus musculus
ggaggtggtgatagccggtat

SEQ ID NO: 10 - DNA, 21 bp, Mus musculus
tgggtaatccatagagcccag

SEQ ID NO: 11 - DNA, 22 bp, Homo Sapiens
tctagctcctataccaccacca

SEQ ID NO: 12 - DNA, 22 bp, Homo Sapiens
tcgtctccaacttatctcctcc

SEQ ID NO: 13 - DNA, 22 bp, Mus musculus
ttcttgcgatacactctggtgc

SEQ ID NO: 14 - DNA, 22 bp, Mus musculus
cgggattgaatgttcttgtcgt

SEQ ID NO: 15 - DNA, 19 bp, Mus musculus
tgacccggctattccgtga

SEQ ID NO: 16 - DNA, 21 bp, Mus musculus
ctgggctgagcaatacagttc